(19) United States
(12) Patent Application Publication
(10) Pub. No.: US 2012/0107353 A1
(43) Pub. Date: May 3, 2012

(54) COMPOSITION

(75) Inventors: Bror Morein, Uppsala (SE); Karin Lövgren Bengtsson, Uppsala (SE)

(73) Assignee: ISCONOVA AB, Uppsala (SE)

(21) Appl. No.: 13/383,017

(22) PCT Filed: Jul. 9, 2010
(86) PCT No.: PCT/SE2010/050798

§ 371 Date: Jan. 10, 2012

Related U.S. Application Data
(60) Provisional application No. 61/224,479, filed on Jul. 10, 2009.

(51) Int. Cl. A61K 39/12; A61K 39/02; A61P 37/04; A61K 39/00

(52) U.S. Cl. 424/204.1; 424/184.1; 424/234.1; 424/265.1; 424/274.1

(57) ABSTRACT

The invention relates to a composition comprising at least one ISCOM complex and at least one internal antigen which is not a surface antigen and not in the form of a part of a whole micro-organism. The internal antigen may be a nucleoprotein or presented as a member of the group of components obtained after disintegrating a micro-organism. The ISCOM complex may be an ISCOM or ISCOM matrix complex. The composition may also comprise non internal antigens. The invention also elates to the composition for use as an immune stimulating medicine or vaccine, especially for use in eliciting T cell respond including CTL respond. The invention also relate to a composition comprising at least one ISCOM complex for use as an immune stimulating or immune modulating medicine or vaccine for the stimulation of dendritic ceils in elderly. Further, the invention relates to a process for preparing a composition wherein a saponin, cholesterol and a lipid are mixed with a lysed cell suspension of cells and solubilising agent without removal of any cell components, where after the solubilising agent is removed or diluted. It also relates to a kit.

The present invention relates to a composition comprising at least one ISCOM complex and at least one internal antigen which is not a surface antigen and not in the form of a part of a whole micro-organism. It further regards to the composition for use as an immune stimulating medicine or vaccine, especially for use in eliciting T cell respond including CTL respond. It also regards a composition for use as an immune stimulating medicine or vaccine for low responders.

The invention also relates to a composition comprising at least one ISCOM complex for use as an immune stimulating or immune modulating medicine or vaccine for the stimulation of dendritic cells in elderly.

Further, the invention relates to a process for preparing the composition and a kit.

The invention encompasses ISCOM/ISCOM-Matrix formulations that are used to enhance and broaden the immune responses to enhance the level and/or quality of immune responses to accessible vaccine antigens or by revealing antigens hidden in the whole micro-organisms/viruses and to broaden the immune responses to non-surface antigens revealed by disintegration of intact micro-organisms and to evade immune suppression exerted by an intact microorganism including viruses. It also includes stimulation of specific antibodies in various subclasses, cell mediated immune responses including Th1 and Th2 and cytotoxic T cell responses i.e. balanced immune responses to achieve immune protection. It also includes fast immune response for required situations. The invention also includes the use of ISCOM/ISCOM-Matrix adjuvant system to turning non-responding individuals to immune responders.

### PRIOR ART

Adjuvants are used to enhance vaccines either for prophylaxes or therapy. However, present vaccines need improved effect to be efficient in various vaccine fields. Examples of vaccine that may need improved effects are Rabies, RSV and influenza vaccines. These need improvement by reducing the number of non- and low-responders. Influenza virus has a strong tendency to evade the immune response evoked by vaccines of today by escape mutants. Rabies virus is another virus, which should function given after infection. For respiratory syncytial virus (RSV) an efficient vaccine is lacking mainly causing problems in infants and elderly where a vaccine with an alternative capacity is required. Other target vaccines are in the Herpes virus family.

A special use for a vaccine is in connection with rabies virus infections. For human use, the rabies vaccine is mostly applied after a suspected infection with the rabies virus i.e. posts infection. To be effective after infection the vaccine has to induce a fast and potent immune response to precede the disease, in the case of rabies, to prevent death.

An efficient adjuvant, which concerns most kinds of vaccines, shall not only induce high levels of immune responses, but also high quality responses including antibody and cell mediated immune responses i.e. right type of immunity to achieve immune protection. The specific immunity means immune response(s) directed to a specific component or components of the agent against which the vaccine is intended. The vaccine must, therefore, contain and expose that component(s), i.e. the vaccine antigen(s), which might be a protein a part of protein or a carbohydrate generally linked to a protein and then named glycoprotein. The antigens selected for the conventional vaccines and used in conventional vaccines for induction of immune protection are generally surface proteins, which are exposed on the surface of the pathogen being target for the vaccine.

With regard to protective antibodies i.e. inducing immune protection those are generally exposed on the surface of the agent. For viruses protective antibodies are often characterized virus neutralizing (VN) antibodies. There are other important immune mechanisms than antibodies, namely cell mediated immunity (CMI) including cytotoxic T-cells (CTL) with capacity to kill infected cells being particularly important for protection against virus infections but also important against other intracellular or optionally intracellular pathogens/parasites. Thus, CMI might be even more important than antibodies for protection against intracellular parasites. Of notion is that CMI against internal/intracellular protective antigens/epitopes may induce a broader immunity than antibodies that covers immune protection against specific variants/subtypes of various pathogens. Thus, CMI might cover cross protection to other variants/isolates than the antibody arm of the immune system covers.

EP 0 109 942 B1 discloses ISCOM complexes produced by solubilizing microorganisms creating a mixture of solubilizing agent and cell or microorganism fragments. Charged monomeric antigenic proteins with hydrophobic regions are complex bound to the solubilizing agent. By separating the charged monomeric antigenic proteins from the solubilizing agent in the presence of, or by directly transferring them to, one or more glycosides with hydrophobic and hydrophilic regions presented in a concentration of at least the critical micelle concentration an immunogenic complex is produced. The rest of the fragments are removed before the complex according is produced, while it is being produced, or afterwards. These ISCOM complexes will mainly comprise surface antigens or membrane antigens that are hydrophobic and no internal antigens.

### SUMMARY OF THE INVENTION

The present invention reveals internal antigens besides externally exposed antigens of disease provoking microorganisms (pathogen) and make them immunogenic by use of ISCOM Matrix formulations. The pathogen might be a whole (complete microorganism including viruses) or disintegrated microorganism. The whole microorganism may not expose the internal antigens to is the immune system that evokes immune responses unless the adjuvant according to the invention is present.

To broaden the immune response to include internal antigens to participate in the immune protection may also contribute to increase fast immune protection particularly after post exposure use and also to long lasting immunity.

Besides increasing efficacy including antigen sparing of external antigens quality of immune responses the present invention relates to a composition comprising at least one ISCOM complex and at least one internal antigen which is not a surface antigen and not in the form of a part of a whole micro-organism. It further regards to the composition for use as an immune stimulating medicine or vaccine, especially for use in eliciting T cell respond including CTL respond.

Vaccines are mostly based on whole microorganisms or subunits that promote immune responses including both antibody and T cell responses against surface structures. Alternatively, the vaccine antigens are subunits i.e. most often the surface proteins, but also internal/intracellular proteins or even non-structural proteins that might be expressed in cellular vectors. The latter are then used to evoke T cell responses since antibodies do not interact with internal proteins and can, therefore, not mediate immune protection. In the present invention the internal proteins are revealed by disintegrating the agent e.g. the microorganism to expose other proteins/antigens that are immunogenic and of immune protective value. The disintegration is e.g. done by solubilization of the agent/microorganism.

Products from the ISCOM technology are used to enhance the immunogenicity of the accessible antigens i.e. surface antigens and the antigens revealed by the disruption of the agent against which the vaccine is prepared.

The present invention is addressing the advantage of, besides evoking immune response to the antigens covered by conventional vaccines, also to cover internal antigens. These may be nucleoproteins or intracellular non-structural proteins of the agents including viruses and intracellular pathogens that might be revealed e.g. in cells used as expression vectors for immune stimulation. Thus, a broadened effect compared to or in contrast to conventional vaccine techniques in the field is obtained by making internal antigens and intracellular antigens accessible by disrupting the pathogen including cells or by making those available for immune induction by use of the whole microorganism. These internal antigens are used together with ISCOM formulation and adapted ISCOM techniques as adjuvant to enhance the CMI against such internal/intracellular antigens resulting in broadened immune response(s) and in immune protection. The invention is also targeting the vaccine production process by using different methods for formulating the ISCOM components i.e. in the same sequence as disrupting the pathogen or using preformed ISCOM Matrix when preparing the final adjuvanted vaccine. Thus, the invention is improving vaccines by making increased number of protective vaccine antigens available i.e. broadening the immune response and by stimulation of CMI. In this improved antigen formulation the adjuvant is playing an essential role by enhancing the CMI arm but also the antibody mediated immunity of the immune system.

ISCOM—the classical ISCOM is the ISCOM-particle with antigen(s) included, physically inserted into the ISCOM structure. The term “ISCOM” is also used in a more general sense Including both ISCOM and ISCOM Matrix type of preparations.

ISCOM Matrix—the classical Matrix particle being an ISCOM without inserted antigens.

Man M—A combination of Matrix A (made from saponin Fraction A) and Matrix-C (made from saponin Fraction-C) particles.

ISCOM Immune response covers also immune responses.

Subunit/Component(s) are antigen, part of antigen, antigenic determinant/epitope including protein/peptide carbohydrate moieties optionally linked to i.e. glycoprotein.

Protective immunity means alternatively immune protection alternatively immune defense or defense.

Immune stimulation includes stimulation of immune protection.

Variants, includes subtypes, types, subzero-types and sero-types are of similar species of microorganism/virus for which a broadened immune responses are intended to involve in immune protection.

Vaccine antigen includes whole microorganism/virus, subunit, antigen determinant, epitope etc. intended for induction of any type of immune response.

Agents include any type of microorganism/virus products of microorganisms like toxins and allergens.

Immune defense includes defense.

Cross protection means immune protection to additional variants, subtypes, types, subzero-types and sero-types are of similar species of microorganism/virus that conventional vaccines do not cover by immune protection.

Identification may be done by e.g., serological testing or nucleotide typing or any other state-of-the-art method.

Pathogen means any type of microorganism/virus part thereof e.g. toxin or allergen.

Parasite might include any type of microorganism/virus e.g. virus is an intracellular parasite.

Internal/intracellular means components in virus, microorganism, bacterial cells, eukaryote cells including mammalian cells, insect cells or yeast cells.

Responder(s) are individual(s) responding to immunization/vaccination, while non-responder(s) are individual(s) not responding to immunization/vaccination.

Disruption includes disintegration or any word that means breaking cell or virus membranes or taking viruses or cells apart.

Broaden immune response means compared to conventional vaccine formulation to include additional variants, subtypes, types, subzero-types and sero-types that are of similar species of microorganism/virus for which a broadened immune response(s) are intended to involve in immune protection.

### FIGURE LEGENDS

FIG. 1. Matrix M and ISCOM formulated rabies vaccine induces high titers of antigen specific antibodies of both IgG1 and IgG2a subclasses already after primary immunization. 1A. IgG1, primary response. 1B. IgG2a, primary response. Matrix M consisted of 83% Matrix A and 17% Matrix C.

FIG. 2. Matrix M and ISCOM formulated rabies vaccine induces higher titers of antigen specific IgG2a antibodies than the corresponding formulations without Matrix M. 2A. IgG1, secondary response. 2B. IgG2a, secondary response. Matrix M consisted of 83% Matrix A and 17% Matrix C.

FIG. 3. Virus neutralizing (ELISA) serum antibody response in mice is detected already after priming in Matrix M adjuvanted vaccine and is further enhanced after booster. 3A. Primary response. 3B. Secondary response after booster. Matrix M consisted of 83% Matrix A and 17% Matrix C.

FIG. 4. Rabies virus-neutralizing antibody titers (OIE approved serum neutralization test) in Grey Fox. Eight foxes per group, age 2-4 years, were vaccinated days 0 and 28 with (Group 1) WRV+Al(OH)₃; (Group2) WRV+Matrix M; (Group 3) Commercial adjuvanted Rabies vaccine (Group 4) Non-vaccinated controls. Serum samples were taken at days 0, 21, and 42. Matrix M consisted of 83% Matrix A and 17% Matrix C.

FIG. 5. Protein profiles in SDS-PAGE of (A) Whole Rabies Virus (WRV) and (B) Disintegrated Rabies Virus (DiRV) antigen formulations used for immunization of mice. 5A WRV of Pitman Moore strain. 5B DiRV of TS80 strain.

FIG. 6. Western blot analysis of sera from mice immunized with WRV (lane 2 and 3) or DiRV (lane 4 and 5) with (lane 3 and 5) and without (lane 2 and 4) Matrix-M adjuvant. 6A, blotted against WRV of Pitman Moore strain. 6B, blotted against TS80 strain. Matrix M consisted of 83% Matrix A and 17% Matrix C.

FIG. 7. IgG1 and IgG2a response to Rabies virus vaccines with or without Matrix M addition. 7A, primary IgG1 response. 7B, primary IgG2a response. Matrix M consisted of 83% Matrix A and 17% Matrix C.

FIG. 8. IgG1 and IgG2a responses to Rabies virus vaccines with or without Matrix M addition. 8A. IgG1, secondary response. 8B. IgG2a, secondary response. Matrix M consisted of 83% Matrix A and 17% Matrix C.

FIG. 9. Virus neutralizing (ELISA) antibody response in mice is detected already after priming in Matrix M adjuvanted vaccine and is further enhanced after booster. 9A. Primary response. 9B. Secondary response.

FIG. 10. IL-2 response after re-stimulation of spleen cell from mice vaccinated with WRV or DiRV with or without Matrix-M adjuvant. BALB/c mice were immunized twice s.c. with DiRV or Matrix M adjuvanted DiRV, WRV or Matrix M adjuvanted WRV. Spleen cells collected 14 days after the second immunization were re-stimulated with purified rabies virus N-protein or WRV for 72 hours. IL-2 was measured in spleen cell supernatants using CBA (cytometric bead array).

FIG. 11. IFN-γ response after re-stimulation of spleen cell from mice vaccinated with WRV or DiRV with or without Matrix M adjuvant. Balb/c mice were immunized twice s.c. with DiRV or Matrix M adjuvanted DiRV, WRV or Matrix M adjuvanted WRV. Spleen cells collected 14 days after the second immunization were re-stimulated with purified rabies virus N-protein or WRV for 72 hours. IFN-γ was measured in spleen cell supernatants using CBA (cytometric bead array).

FIG. 12. IL-4 response after re-stimulation of spleen cell from mice vaccinated with WRV or DiRV with or without Matrix M adjuvant. Balb/c mice were immunized twice s.c. with DiRV or Matrix M adjuvanted DiRV, WRV or Matrix M adjuvanted WRV. Spleen cells collected 14 days after the second immunization were re-stimulated with purified rabies virus N-protein or WRV for 72 hours. IL-4 was measured in spleen cell supernatants using CBA (cytometric bead array).

FIG. 13. IL-5 response after re-stimulation of spleen cell from mice vaccinated with WRV or DiRV with or without Matrix M adjuvant. Balb/c mice were immunized twice s.c. with DiRV or Matrix M adjuvanted DiRV, WRV or Matrix M adjuvanted WRV. Spleen cells collected 14 days after the second immunization were re-stimulated with purified rabies virus N-protein or WRV for 72 hours. IL-5 was measured in spleen cell supernatants using CBA (cytometric bead array).

FIG. 14. Antibody responses (ELISA) in Balb/c mice to D-Flu antigens with or without Matrix M adjuvant following one (A, C) or two (B, D) s.c. immunizations four weeks apart. A and B, Primatry and secondary IgG1 response. C and D, Primary and secondary IgG2a response. The antibody responses are measured against H1 N1 component (A/New Caledonia/20/99) in the vaccines. Matrix M consisted of 90% Matrix A and 10% Matrix C.

FIG. 15. Adjuvant effect of Matrix M on immunization with RSV, enhancement of VN antibody levels in serum. Cotton Rats were vaccinated with; 1 μg (filled circles) or 5 μg (filled squares) of DiRSV adjuvanted with Matrix M. The Matrix M consisted of 83% Matrix A and 17% Matrix C. Two control groups were included; triangles top up were infected with live virus at day 0 and triangles top down were untreated controls until challenge at day 46.

FIG. 16. Matrix-M adjuvanted DiRSV induces immune protection in cotton rat human RSV model by reduction of virus replication in upper respiratory tract and lungs. Grey filled squares represent the response in Nasal washes whereas the dotted squares represent the response in Lung lavage.

FIG. 17. Proportion (%) of CD 83 cells following stimulation with Matrix M adjuvant in an ex vivo human DC model. The cells were stimulated with 200, 100 and 10 mg of Matrix A; 10, 1 and 0.1 mg of Matrix C and with 100, 10 and 1 mg of Matrix M. The matrix M consisted of 87% Matrix A and 17% Matrix C.

FIG. 18. Proportion (%) of CD 86 cells following stimulation following stimulation with Matrix adjuvant in an ex vivo human DC model. The cells were stimulated with 200, 100 and 10 mg of Matrix A; 10, 1 and 0,1 mg of Matrix C and with 100, 10 and 1 mg of Matrix M. The matrix M consisted of 87% Matrix A and 17% Matrix C.

FIG. 19. Proportion (%) of CD83 positive cells after treatment of monocytes from elderly volunteers (see Materials and Methods). N=10 elderly individuals

Day 0 Untreated monocytes

iDCs Immature DCs obtained after culture of monocytes for 5 days with GM-CSF and IL-4

MMD 1.0 Matrix M 10 pg +DiRSV 1 μg

MMD 0.5 Matrix M 10 pg +DiRSV 0.5 μg

LPS LPS 1 μg used as positive control

MEDIUM Medium control

FIG. 20. Proportion (%) of CD86 positive cells after treatment of monocytes from elderly volunteers (see Materials and Methods). N=10 elderly individuals

Day 0 Untreated monocytes

iDCs Immature DCs obtained after culture of monocytes for 5 days with GM-CSF and IL-4

MMD 1.0 Matrix M 10 μg+DiRSV 1 μg

MMD 0.5 Matrix M 10 μg+DiRSV 0.5 μg

LPS LPS 1 μg used as positive control

MEDIUM Medium control

FIG. 21. An ISCOM adjuvanted Neospora vaccine formulation induced potent antibody response in calves. All animals were challenged by infection with live Neospora at week 11.

Group A—Calves immunized i.v. with live Neospora at day 0.

Group B—Calves were immunized s.c. with 500 mg disintegrated Neospora formulated with Matrix Q at days 0 and 42.

Group C—Calves were immunized s.c. with 500 mg disintegrated Neospora at days 0 42.

Group D—Control calves given 750 mg Matrix Q alone

Group E—Control calves given PBS (vaccine diluent)

FIG. 22. An ISCOM Matrix adjuvanted Neospora vaccine formulation induced potent IFN-γ response in calves that was not down regulated by a subsequent infection

Group A—Calves immunized i.v. with live Neospora at day 0.

Group B—Calves were immunized s.c. with 500 mg disintegrated Neospora formulated with Matrix Q at days 0 and 42.

Group C—Calves were immunized s.c. with 500 mg disintegrated Neospora at days 0 42.

Group D—Control calves given 750 mg Matrix Q alone

Group E—Control calves given PBS (vaccine diluent)

FIG. 23. Kinetics of mean IgG levels in serum (A) and milk (B) of Heifers immunized with S.A. Bacterin (whole killed bacteria) adjuvanted with Matrix Q or Al(OH)₃. Serum samples and milk sera were diluted 1/5000 and 1/500 respectively in PBS for ELISA.

FIG. 24. Kinetics of mean IgG serum titers of experimental groups vaccinated with two different formulations, S.A. Bacterin and S.A. Lysate. A group given placebo (vaccine diluent) was included. The sera were analyzed against Bacterin (FIG. 24A) or Bacterial Lysate (FIG. 24B).

### DETAILED DESCRIPTION OF THE INVENTION

The present invention reveals internal antigens besides externally exposed antigens of disease provoking microorganisms (pathogen) and make them immunogenic by use of ISCOM Matrix formulations. The pathogen might be a whole (complete microorganism including viruses) or disintegrated microorganism. The examples show that disintegration reveals hidden antigens. Together with an ISCOM formulation e.g. ISCOM or ISCOM matrix the “hidden” antigens are recognized because small amounts of antigens are enough for induction of immune response.

A whole microorganism may not expose the internal antigens to the immune system that evokes immune responses unless the adjuvant according to the invention is present. It is believed that the small amount of antigens are (might be) revealed because a small proportion of the microorganism is spontaneously disintegrated. In Example 7b it is obvious that disintegration reveals internal antigens of Staphylcoccus aureus but also that the ISCOM formulation improves the immune response if the whole bacterin is used instead of a disintegrated bacteria.

The invention relates to a composition comprising at least one ISCOM complex and at least one internal antigen which is not a surface antigen.

According to one embodiment the internal antigen is not in the form of a part of a whole microorganism.

According to one other embodiment the internal antigen is in form of a whole microorganism. Therefore, the invention also relates to whole pathogens where the ISCOM complex increase the immunogenicity of whole pathogens/microorganisms considerably over present available vaccines including capacity to enhance internal antigens to evoke immune responses.

The internal antigen may include (be one or more) nucleoproteins, polymerase or, it may be a member of the group of components obtained after disintegrating a whole micro-organism.

Thus, the composition according to the invention may comprise a whole or disintegrated whole micro-organism and at least one ISCOM complex. Such disintegrated slurry of whole microorganism will contain and expose internal antigenic components which may be a protein, a part of protein or a glycoprotein comprising a carbohydrate linked to a protein.

The internal antigen may be a purified internal antigen, which is not a surface epitope or surface antigen and which is not a membrane protein with surface epitope(s). The internal antigen may be an antigen which is not reachable from the surface of the micro-organism. It could be an internally faced antigenic part of a membrane protein.

Disintegration may be performed with enzymes, detergents, solubilizing agents or by physical force, e.g. by pressure or mechanically e.g. with beads e.g. heavy metal beads or small glass beads, high pressure or ultrasonic methods. Examples of useful solubilizing agents for disintegration are mentioned below.

Disintegration is conventionally used in the formulation of influenza virus to eliminate side effects, such as headache, muscle pains and nausea partly due to high levels of IFN-y. In the present invention that is not purpose as described for influenza virus but safety for another effect i.e. disintegration of a pathogen is the most secure way to prohibit proliferation by killing the infectious particles. Moreover, there are methods to confirm that no complete particles are present in a suspension of microorganism/viruses, e.g. by microscopy en EM (electron microscopy).

The invention also regards a composition comprising a slurry of one or more disintegrated microorganisms and at least one ISCOM complex.

Solubilising agents may be used that are compatible with pharmaceutical use or use in vaccines and which need not be deleted after integration.

Therefore, the invention also regards a composition, wherein the least one internal antigen is a member of the group of components obtained after disintegrating a micro-organism with a solubilising agent. This is a composition comprising at least one solubilising agent, at least one disintegrated type of micro-organism and at least one ISCOM complex.

The invention may further comprise other added antigens e.g. rDNA or synthetically produced antigens or any of the above mentioned antigens which may be added to the compositions.

The solubilising agent in the composition may have been diluted 2-100 times after the disintegration to make up a suitable vaccine composition.

The ISCOM and the ISCOM matrix are adjuvant components in the composition.

According to one embodiment, the ISCOM complex is an ISCOM comprising at least one saponin, at least one lipid and at least one type of antigen substance. The lipid is at least a sterol such as cholesterol and optionally also phosphatidyl choline. This complexes may also contain one or more other immunomodulatory (adjuvant-active) substances, and may be produced as described in EP 0 109 942 B1, EP 0 242 380 B1 and EP 0 180 564 B1. Moreover, a transport and/or passenger antigen may be used, as described in EP 9600647-3 (PCT/SE97/00289).

in another embodiment of the invention, the immunogenic complex constitutes an ISCOM-matrix complex and said ISCOM-matrix complex is used together with one or more antigens intended to elicit specific immune response to included antigen(s) and/or said ISCOM-matrix complex and antigens are separate entities (units) intended to be administered in mixture or separately. An ISCOM matrix comprises at least one glycoside and at least one lipid. The lipid is at least a sterol such as cholesterol and optionally also phosphatidyl choline. The ISCOM complexes may also contain one or more other immunomodulatory (adjuvant-active) substances, not necessarily a saponin, and may be produced as described in EP 0 436 620 B1.

The ISCOM formulation or the components thereof i.e. the saponin and the lipid e.g. the phospholipid and the cholesterol may be added either during the disintegration process or after completed disintegration.

The ISCOM Matrix formulation might be supplemented as a complete adjuvant formulation i.e. ISCOM Matrix.

The adjuvant components are preferentially quillaja saponins, crude preparations or purified Fractions not excluding other saponins like ginseng saponins or fractions thereof e.g. other adjuvant molecules like LPS/lipid A.

The sapoin may be chosen from Quillaja Saponaria Molina fraction A, fraction B, fraction C of Quillaja Saponaria Molina, a raw fraction of Quillaja Saponaria Molina such as spicoside, fraction Q, VAC, QA 1-23. When prepared as described herein, Fractions A, B and C of Quillaja Saponaria Molina each represent groups or families of chemically closely related molecules with definable properties. The chromatographic conditions under which they are obtained are such that the batch-to-batch reproducibility in terms of elution profile and biological activity is highly consistent.

The term “ one saponin fraction from Quillaja Saponaria Molina.” is used throughout this specification and in the claims as a generic description of a semi-purified or defined saponin fraction of Quillaja Saponaria or a substantially pure fraction. It is important that the fraction does not contain as much of any other fraction to negatively affect the good results that are obtained when the mixtures of ISCOM or ISCOM matrix comprising essentially one fraction is used. The saponin preparation may, if desired, include minor amounts for example up to 40% by weight, such as up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight up to 0.1% by weight of other compounds such as other saponins or other adjuvant materials.

The saponin fractions A, B and C according to the present invention are as described in WO 96/11711, the B3, B4 and B4b fractions as described in EP 0 436 620; the fractions QA1-23 are as described in EP 0 3632 279 B2. The fractions QA-1-2-3-4-5-6-7-8-9-10-11-12-13-14-15-16-17-18-19-20-21, 22 and 23 of EP 0 3632 279 B2, especially QA-7, 17-18 and 21, may be used. They are obtained as described in EP 0 3632 279 B2, especially on page 6 and in Example 1 on page 8 and 9.

Any type of raw fractions of saponins from Quillaja Saponaria Molina may be used. A raw fraction of Quillaja Saponaria Molina is any saponin fraction thereof substantially freed from other non saponin components. Also partly purified saponin fraction, obtained by selection or removal of defined materials may be used. Reversed phase fractions of the Quil A may also be used. Such raw fraction wherein the saponins are not separated from each other may be produced by modern separating techniques, e.g., chromatography or extraction procedures. Examples of raw saponin fractions from Quillaja Saponaria Molina are fraction Q and Q-VAC and Spicoside and any fraction comprising fractions A, B and C substantially freed from other non saponin material; any fraction comprising QS 1, 2, 3-and up to QS23 (also named QA 1-23) substantially freed from other non saponin material. Q-VAC is commercially available (Nor-Feed, AS Denmark), as is Quillaja Saponaria Molina spicoside. Examples of raw fractions of Quillaja Saponaria Molina are described in WO 9003182 and K Dalsgaard: Saponin Adjuvants III, Archiv fur die Gesamte Virusforschung 44, 243-254 (1974).

Fractions A, B and C described in WO 96/11711 are prepared from the lipophilic fraction obtained on chromatographic separation of the crude aqueous Quillaja Saponaria Molina extract and elution with approximately 70% acetonitrile in water to recover the lipophilic fraction. This lipophilic fraction is then separated by semi preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluated at approximately 49% acetonitrile.

According to one embodiment a raw fraction of saponins is used.

According to another embodiment a raw fraction of saponins may be used together with any other purified saponin fraction, e.g. the different saponin fractions mentioned above.

According to one embodiment, there is provided an immunogenic complex for use according to the invention, comprising from 5-99% by weight of one fraction, e.g. fraction A of Quillaja Saponaria Molina and the rest up to 100% of weight of another fraction e.g. a raw saponin fraction or fraction C of Quillaja Saponaria Molina counted on the weight of fraction A and fraction C.

According to another embodiment, there is provided an immunogenic complex for use according to the invention, comprising from 40% to 99% by weight of one fraction, e.g. fraction A of Quillaja Saponaria Molina and from 1% to 60% by weight of another fraction, e.g. a raw saponin fraction or fraction C of Quillaja Saponaria Molina counted on the weight of fraction A and fraction C.

According to yet an embodiment, there is provided an immunogenic complex for use according to the invention, comprising from 70% to 95% by weight of one fraction e.g. fraction A of Quillaja Saponaria Molina and from 30% to 15% by weight of another fraction, e.g. a raw saponin fraction or fraction C of Quillaja Saponaria Molina counted on the weight of fraction A and fraction C.

In one embodiment, there is provided an immunogenic complex for use according to the invention, wherein the saponin fraction from Quillaja Saponaria Molina is chosen from any one of QA 1-22.

In one embodiment, the composition for use according to the invention comprises at least two different immunogenic complexes chosen from ISCOM complexes and/or ISCOM-matrix complexes, each individual complex comprising one saponin fraction from Quillaja Saponaria Molina, wherein the saponin fraction in one complex is different from the saponin fraction in the other complex. Thus, one type of substantially pure saponin fraction or a raw saponin fraction may be integrated into one ISCOM or ISCOM matrix complex or particle and another type of substantially pure saponin fraction or a raw saponin fraction may be integrated into another ISCOM or ISCOM matrix complex or particle. A composition or vaccine may comprise at least two types complexes or particles each type having one type of saponins integrated into physically different particles.

Mixtures of ISCOM and for matrix may be used in which one saponin fraction Quillaja Saponaria Molina and another saponin fraction Quillaja Saponaria Molina are separately incorporated into different ISCOM complexes or matrix. Any combinations of weight % of the different ISCOM complexes based on their content of one fraction, e.g. fraction A and another fraction, e.g. any raw saponin fraction or fraction C of Quillaja Saponaria Molina respectively may be used. The mixtures may comprise from, 0,1 to 99,9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60%, by weight, 50 to 50% by weight, 55 to 45% by weight, 60 to 40% by weight, 65 to 35% by weight, 70 to 30% by weight, 75 to 25% by weight, 80 to 20% by weight, 85 to 15% by weight, 90 to 10% by weight, 95 to 05% by weight, 50 to 99% by weight, 60 to 90% by weight, 70 to 90% by weight, 70-99 by weight, 75 to 85% by weight%, of ISCOM complexes comprising one saponin fraction, e.g. fraction A of Quillaja Saponaria Molina and the rest up to 100% in each case of interval of ISCOM complexes comprising another saponin fraction, e.g. any raw fraction, e.g. fraction C of Quillaja Saponaria Molina, counted on the content of the sum fractions A and C of Quillaja Saponaria Molina in the ISCOM complexes.

The above figures relate to combinations of any saponin fraction of Quillaja Saponaria Molina integrated into the same of different ISCOM or ISCOM matric complex or particle e. g. fraction A in combination with any of fractions C, B and a raw fraction e.g. fraction Q.

By combining ISCOM complexes and/or ISCOM Matrix complexes comprising different fractions of Quillaja Saponaria Molina it is possible to produce compositions that are less toxic. Hence, in one embodiment, the composition for use according to the invention comprises fraction A in combination with at least one of fractions C and Q, in the same or different ISCOM complexes and/or ISCOM-matrix complexes.

According to one embodiment a combination of fraction A and fraction C is used in the same or in different particles. Such combinations may consist of 30-70%, 80-99%, 80-95%, 80-92%, 83-99%, 83-95%, 83-92%, of fraction A and the rest up to 100% of fraction C based on the weight of the saponin fractions in the same or in different particles. When in different particles these saponin compositions are called Matrix M.

In another embodiment, there is provided a composition for use according to the invention, further comprising at least one other adjuvant. This further andjuvant may be a saponin fraction from Quillaja Saponaria Molina, which may not bound to or integrated into the immunogenic complex. Such other adjuvants and saponines or glucosides that are not integrated into the ISCOM or ISCOM matrix may be mixed into the composition.

Examples of other adjuvants that can be incorporated in the ISCOM and ISCOM matrix are any adjuvant, natural or synthetic, with desired imunomodulatory effect, e.g. muramyl dipeptide (MDP)-derivatives, such as fatty acid, substituted MDP, threonyl analogues of MDP; DDA, poly anions such as Dextran sulphate, lipopolysaccarides such as saponins (other than Quil A). (“Future prospects for vaccine adjuvants”, Warren, H. S. (1988) CRC Crit. Rev. Immunol. 8:2, 83-101; “Characterisation of a non-toxic monophosphoryl lipid A”, (1987) Johnson, A. G. et al, Rev. Infect. Dis. 9:5, 5512-5516; “Developmental status of synthetic immunomodulators”, Berendt, M. J. et al (1985), Year Immunol. 193-201; “Immunopotentiating conjugates”, Stewart-Tull, D. E., Vaccine, 85, 3:1, 40-44).

The internal antigen may be chosen from internal components in micro-organisms such as virus, bacteria, parasites, yeast cells, eukaryotic cells, including mammalian cells, insect cells.

Examples of bacteria are e.g. Escherichia, Staphylococci, e.g. Staphylococcus aureus and coagulase negative Staphylococcus, Streptococci e.g. Streptococcus pyogenes, Streptococcus dysgalactiae, Streptococcus agalactiae and Streptococcus uber is Haemaophiius, e.g. H. influenzae, Bordetella, e.g. B. pertussis, Vibrio, e.g. V. cholerae, Salmonella, e.g. S. typhi, S. paratyphi, preferably adherence factor in Coli, e.g. pili K 88 and porin protein in e.g. Salmonella or outer membrane proteins from B. pertussis and Neisseria meningitidis.

Examples of usable viruses with envelopes are Orthomyxoviridae such as influenza A, B, C, RSV), Paramyxoviridae, especially measles virus, mumps virus, parainfluenza 1, 2, 3 and 4 viruses, canine distemper virus and rinderpest virus, Rhabdoviridae, especially rabies virus, Retroviridae, especially feline leukemia virus and bovine leukemia virus, Herpesviridae, especially Pseudorabies, Rabies, e.g. Bat Lyssa viruses such as the Duvenhagen strain, Rabies internal N- and P-proteins Coronaviridae, Togaviridae, such as EEE.WEE.VEE (eastern, western and Venezuela equine encephalitis), yellow fever virus, especially bovine virus diarrhea virus, and European swine fever virus Arenaviridae, Poxviridae, Bunyaviridae, Iridioviridae, especially African swine fever virus and among unclassified viruses, and Marburg/Ebola virus.

Examples of non-enveloped viruses with non-hydrophobic proteins are Picornaviridae, e.g. foot-and-mouth disease virus, polio virus, hepatis A virus, Adenoviridae, Parvoviridae, e.g. feline pest virus and swine parvovirus, Reoviridae, e.g. Rotavirus., Circovirus Examples of mycoplasma are M. pnemoniae, mycoides, bovis, suis, orale, salvarium, hominis and fermentans.

Examples of parasites which can be used according to the invention are Protoza, such as Toxoplasma, e.g. Toxoplasma gondii, Plasmodium, e.g. Plasmodium vivax, malariae, falciparium, Teileria parvum ovale and Filaroidae, preferably Parafilaria and Onchocerca, Entamoeba histolytica, anaplasma of various types, Schistosoma such as Schistosoma haematobium, mansoni, japonicum, and Trypanosoma, e.g. Trypanosoma gambiense, brusei or congolesi and Neospora caninum.

According to one embodiment the internal antigens derive from RSV virus, Rabies virus, influenza virus, Neospora or Staphylococcus aureus.

According to another embodiment the antigens are in form of disintegrated whole cells e.g. from RSV virus, Rabies virus, influenza virus, Neospora or Staphylococcus aureus, which may be present in the medium used for disintegration, which medium may be diluted as mentioned herein.

According to another embodiment the antigens are harbored in the whole microorganism. Thus, the composition may further also comprise whole micro-organisms which may be live and attenuated. These are not disintegrated.

According to one embodiment the antigens are in form of whole cells e.g. from RSV virus, Rabies virus, influenza virus, Neospora or Staphylococcus aureus.

The internal antigen may be integrated into an ISCOM complex, mixed with an ISCOM matrix complex or mixed with an ISCOM complex or coupled on to an ISCOM complex or ISCOM matrix complex. When the internal antigen or whole microorganisms are mixed with an ISCOM complex, the ISCOM complex may comprise another antigen, which could be but need not be an internal antigen and which could be a surface antigen.

The invention further relates to the use of internal Rabies virus antigens, such as the N- and P proteins, disintegrated Rabies virus cells with or without solubilisation agent, which may be diluted and to the use of whole Rabies virus which may be attenuated. These may be mixed with ISCOM or ISCOM matrix as mentioned above or integrated into ISCOM complex. Optionally, cells used for antigen production as for instance the insect cell e.g. the Spodeptera frugiperda (Sf9) expresses the vaccine antigen e.g. the nucleoprotein (NP) the non-structural to (NS) protein of rabies virus.

The composition according to the invention may further also comprise non-internal antigens. These may be membrane proteins and determinants exposed on the surface of microorganisms.

The composition may also comprise one or more additives such as pharmaceutically acceptable excipients, carriers and/or diluents.

The formulation of pharmaceutical compositions and vaccines is well known to persons skilled in the art. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional media or agent is incompatible with the active ingredient, use thereof in the pharmaceutical compositions of the present invention is contemplated. Supplementary active ingredients can also be incorporated into the compositions.

The invention also relates to a composition comprising at least one ISCOM complex and at least one internal antigen which is not a surface antigen for use as an immune stimulating medicine or vaccine. Thus, the invention relates to the use of the compositions described herein for the preparation of an immune stimulating medicine or vaccine. The compositions may be used in eliciting T cell respond including CTL respond. Moreover, the compositions are useful as an immune stimulating medicine or vaccine for low responders. Low responders may be sick people, elderly people or juveniles.

It has turned out that the composition according to the invention stimulates the two arms of the immune system. Thus, not only the B-cell derived immune response triggering immune globulins such as IgG are stimulated. Also the T cell derived immune response is triggered by e.g. 1L-2 production as are the TH1 type of cells with e.g. IFN-γ production as well as the TH1 type of cells by e.g. IL-4 and IL-5 production.

The invention also relates to a process for preparing a composition comprising at least one ISCOM complex and at least one internal antigen, which is not a surface antigen and not in the form of a part of a whole micro-organism, characterized in that a saponin, cholesterol and a lipid are mixed with a lysed or disintegrated cell suspension of cells and solubilising agent without removal of any cell components, where after the solubilising agent is removed or diluted.

The compositions may be used for administration to any type of mammal e.g. human or animal species. Examples of animal species to which the formulations according to the invention may be administrated are companion animals such as cats, dogs, horses, birds such as parrots, economical important species such as cattle, e.g. bovine species, swines, sheep, goats.

The compositions may be used for prophylactic treatment as immunomodulating or—stimulating agents or vaccines. Thus for example ISCOM matrix may be used as immunomodulating agent e.g. for elderly. ISCOM matrix together with antigens or ISCOMs may be used as vaccine or as immunomodulating or—stimulating agents e.g. for elderly.

The compositions may be used for post infection treatment as vaccine. ISCOM matrix together with antigens or ISCOMs may be used as vaccine against rabies or influenza post infection.

The pharmaceutical composition could be adapted to oral, parenteral, or topical use and could be administered to the patient as tablets, capsules, solutions, suspensions or the like.

For parenteral administration the compounds according to the invention could be incorporated in a solution or suspension. Parenteral administration refers to the administration not through the alimentary canal but rather by injection through some other route, as subcutaneous, intramuscular,

These preparations could contain at least 0.1% by weight of an active compound according to the. The amount of the active ingredient that is contained in such compositions is so high that a suitable dosage is obtained.

According to one embodiment at least 1 International Unit (I. U.) may be used for humans e.g. at least about 2 I.U. for humans and about 0.5 I.U. may be used for dogs i.e. at least 1 I.U. for dogs. The skilled person would know how to adopt the dose for bigger animals depending on the weight.

Suitable components to formulate ISCOM-Matrix including quillaja saponin components, lipids, and detergent may be added to the disintegrated agent harboring harboring the antigens or added before the disintegration. ISCOM-Matrix or optionally 1SCOMs are formed with integrated antigens when the detergent keeping quillaja components and lipids solubilised is removed. The complex formation is completed by removal of the detergent/disintegration agent or by dilution of the mixture (agent components, detergent/disintegration agent lipid and quillaja components) so that the detergent cannot keep lipids and quillaja components solubilised. Thus, after a completed process the vaccine formulation contains a complex adjuvant formulation with ISCOM Matrix, but optionally also a combination ISCOMs, ISCOM-Matrix and components including the vaccine antigens from the disrupted agent.

The detergents or solubilizing agents may be, but not restricted to, non-ionic, ionic or Zwitterionic detergent or detergent based on gallic acid which is used in excess. Typical examples of suitable non-ionic detergents are polyglycoi esters and polyglycol ethers with aliphatic or arylaliphatic acids and alcohols. Examples of these are alkylpolyoxyethylene ethers with the general formula CnH2n

(OCH2CH2)xOH, shortened to CnEx; alkylphenyl polyoxyethylene ethers containing a phenyl ring between the alkyl group and the polyoxyethy lene chain, abbreviated Cn4 greater than Ex, e.g. Triton(R) X-100 =tert. CsEg.e (octylphenolether of polyethylene oxide), acylpolyoxyethylene esters; acylpolyoxyethylene sorbitane esters, abbreviated Cn sorbitane Ex, e.g. Tween(R)20, Tween(R)80, /3-D-alkyl-glycosides, e.g. j8-D-octyl-glycoside, octyl gicycoside (OG). The glycosides mentioned below can also be used, especially saponin. These are, however, weak detergents and should be used together with other detergents. Typical examples of suitable ionic detergents are cholic acid detergents such as e.g. desoxycholate and cholate. Even conjugated detergents such as e.g. taurodesoxycholate, glycodesoxycholate and glycocholate can be used. Possible Zwitter-ionic detergents are lysolecitin and synthetic lysophospholipids. Even mixtures of the above-mentioned detergents can be used.

Solubilizing can also be performed with alcohols, organic solvents or small amphipathic molecules such as heptane-1,2,3-triol, hexane-1,2,3-triol, acetic acid, mixtures thereof or with detergents.

The invention further relates to a composition comprising at least one ISCOM complex for use as an immune stimulating or immune modulating medicine or vaccine for the stimulation of in immunologically low responders such as non healthy individuals, genetically defect individuals, juveniles, infants or elderly. The invention especially regards the stimulation of dendritic cells of such individuas. ISCOM matrix without or with antigens (e.g. mixed therewith) and ISCOM complexes with antigens may be used for prophylactic treatment, vaccination or post infection treatment of elderly. The dendritic cells may be chosen from CD 80, CD 83, CD 86 and chimocine CCR 7. The elderly may be chosen from the species mentioned above e.g. a human being.

The invention also relates to a kit comprising at least two compartments, wherein one compartment comprises an ISCOM complex comprising at least one internal antigen, which is not a surface antigen and not in the form of a part of a whole micro-organism and the other compartment comprises a prescription for use or wherein the first compartment comprises an ISCOM matrix complex and the other compartment comprises at least one internal antigen, which is not a surface antigen and not in the form of a part of a whole micro-organism.

The at least one internal antigen in the kit may be a member of the group of components obtained after disintegrating a micro-organism.

The details and particulars described above and in the claims and relating to one aspect of the invention apply mutatis mutandis to the other aspects of the invention.

Thus for example all details regarding the saponin fractions relate both to a composition comprising at least one ISCOM complex and at least one internal antigen, which is not a surface antigen, (claim 1), a method for preparing a composition comprising at least one ISCOM complex and at least one internal antigen (claim 21) a kit (claim 22) and to a composition comprising at least one ISCOM complex e.g. ISCOM matrix for use as an immune stimulating or immune modulating medicine or vaccine for the stimulation of dendritic cells in elderly (claim 24).

While the invention has been described in relation to certain disclosed embodiments, the skilled person may foresee other embodiments, variations, or combinations which are not specifically mentioned but are nonetheless within the scope of the appended claims.

All references cited herein are hereby incorporated by reference in their entirety.

The expression “comprising” as used herein should be understood to include, but not be limited to, the stated items.

### Materials and Methods

### Animals

ICR mice supplied by CDC were used for the vaccination and challenge experiments with rabies virus nucleoprotein (Example 1A), Balb/c mice were used for all other mouse studies.

Cotton rats (50-100 gram) kindly provided by Dr. Pedro A. Piedra, (Department of Pediatrics, Baylor College of Medicine, Houston, Texas, USA) were used for the vaccination and challenge experiments with respiratory syncytial virus (Example 2).

Aberdeen Angus calves fifteen five months, from a beef herd located at INTA-Balcarce, Argentina kindly provided by Dr. D. P. Moore1, (Instituto Nacional de Tecnologia Agropecuaria (INTA), Argentina) were used for the vaccination experiments with Neospora caninum tachyzoites (Example 6). Sero-epidemiological data from year 2000 showed a low endemic prevalence of neosporosis in the experimental herd i.e. <1%. The calves were allocated in dog-proof pens, calves were provided with water ad-libidum, standard hay and commercial cattle concentrate.

Twelve primigravid Holstein dairy heifers in the last trimester of gestation belonging to the dairy herds of INTA Rafaela Experiment Station were used in the experiment (Example 7). The animals were injected subcutaneously in the supramammary lymph node area at approximately 40 d and 14 d before expected calving date. Only animals free from S. aureus IMI and with normal udder development at 40 d before expected calving date were included in the trial. Animals were kept under grazing conditions during the experiment.

### Viruses

Rabies viruses: Pitman Moore (PM), ERA, TS80 and Pasteur RIV strains, propagated in VERO, cells were obtained inactivated without adjuvant. Commercial Rabies virus vaccines were obtained from the pharmacy.

Respiratory syncytial virus (RSV): Tracy strain was kindly provided by Dr. Pedro A. Piedra (Department of Pediatrics, Baylor College of Medicine, Houston, Tex., USA). The Long strain of human RS virus (ATCC VR-26) was kindly supplied by Dr Claes Orvell (Huddinge University Hospital, Stockholm). RSV was propagated in HEP-2 or MA 104 cells (ECACC number 85102918).

### Parasites

Neospora caninum tachyzoites of the NC-1 strain was kindly provided by D. P. Moore, Instituto Nacional de Tecnologia Agropecuaria (INTA), Argentina.

N. caninum tachyzoites were propagated in VERO cells monolayer and harvested when 80% of the cells were infected. The tachozoites were released from the cells by sequential passage of the cell monolayer through 21, 23, 25 and 27 gauge needles and subsequently washed in sterile PBS, counted with a haemocytometer and finally used either to formulate the live inoculums or to obtain the disintegrated antigen extract.

### Preparation of Experimental Vaccine Antigens

### Whole Cell Rabies Virus (WCRV)

Inactivated whole rabies virus of Pitman-Moore strain was kindly supplied by Dr Osterhaus (University Rotterdam, The Netherlands).

### Rabies Virus Recombinant Nucleoprotein (N)

Spodeptera frugiperda (Sf9) cells were grown in monolayer (Reid-Sanden F L, Sumner J W, Smith J S, Fekadu M, Shaddock J H, Bellini W J, Rabies diagnostic reagents prepared from a rabies N gene recombinant expressed in baculovirus. J Clin Microbiol. (1990),28(5):858-63). The recombinant plasmid from the rabies Challenge Virus Standard (CVS) strain was prepared at CDC Atlanta (US). Recombinant-virus-infected Sf9-cells were grown for four days at 28° C., and then subjected to disintegration by three freeze-thaw cycles for lysis (see continuation under preparation of N-protein ISCOM formulation).

### Disintegrated Rabies Virus (DiRV)

Rabies virus (ERA-CB2OM or TS80 strain) propagated in VERO cells was purified and concentrated by conventional sucrose density ultracentrifugation. The purified concentrated virus was re-suspended in PBS. The virus antigen concentration was measured by amino-acid analysis (Aminosyraanalys laboratoriet, Uppsala, Sweden). The concentrated rabies virus preparations were killed with 0.2% betapropiolactone (BPL) and further disintegrated by desoxycholate treatment. The pH in the PBS was adjusted to 7.7 and sodium desoxycholate was added to a final concentration of 1.25% and Tween 80 was added to a final concentration of 0.02%. The virus was incubated for 3 hours at 20° C. The various virus preparations and vaccines were quantified in international units IU using a bioassay (EVL, Utrecht, The Netherlands).

### Commercial Rabies Vaccines

Two commercial rabies vaccines (dog vaccines); called “Rabies-High” and “Rabies-Low”, due to their relative content of antigen measured as IU (EVL, The Netherlands).

Purified Rabies N-protein (for in vitro Re-Stimulation of Spleen Cells)

Rabies nucleoproteins (N-protein) were prepared from the disintegrated preparation of TS80. Lyophilized TS80 was dissolved in 1 ml ddW, final concentration 2.76 mg/ml (18 IU/ml, 6.5 IU/mg) according to amino acid analysis. The virus was layered over 10% sucrose for density centrifugation in a Centrikon T-1075 ultracentrifuge, rotor 55:5, at 30 000 rpm for 2 h at 6° C. The supernatant, the sucrose gradient and the pellet were separated. The pellet was dissolved in 200 ml PBS. The protein concentration was estimated using the Bradford assay and SDS-PAGE analysis of purified protein fractions revealed the presence of proteins in the pellet fraction.

### Disintegrated RSV (DiRSV)

Cell culture from HEP-2 cells was collected, sonicated and clarified by centrifugation at 4000 rpm at 4 C for 30 minutes (Sorvall). The supernatant was kept and the pellets discard. The supernatant was then sedimented by centrifugation at 5000 rpm in a GSA rotor (Sorvall) for 24 hours at 4° C. Sedimented virus was re-suspended in 1/500 of the volume in phosphate buffer solution (PBS) (including 2 μg/mL of aprotinin) for 8 hours on ice and soft mixing. Then it was sonicated once more. Concentrated virus was loaded on a sucrose discontinuous gradient (10 to 40% w/v) and centrifugated at 18000 rpm in a SW55Ti rotor (Beckman) for 3 hours. Six fractions and the pellet were analyzed by Western Blot with anti-F antibody (Synagys) and culturing of virus verified the virus containing fractions. Fraction 6 (from top to bottom) and the pellet containing virus and F protein were selected for the next steps. The partially purified virus was disintegrated with 2% of b-octyl glucoside (OG) at 37° C. under soft rotation for 1 hour (in the presence of 2 μg/mL of aprotinin). Disintegrated virus was loaded on a sucrose discontinuous gradient (10 to 30% w/v) and centrifugated at 42000 rpm in a SW55Ti rotor (Beckman) for 1 hour. Five fractions were analyzed for virus and the presence of F protein by Western Blot. The 2nd fraction (from top to bottom) was selected showing high signals for F protein. SDS-PAGE stained with silver nitrate and Western blot showed that fractions had a complex pattern of proteins including most virus proteins it was named disintegrated virus (DiRSV). It also contained cellular proteins.

### Influenza Antigen

Influenza virus (H3N2) was obtained as a commercial non-adjuvanted vaccine.

### Preparation of Live and Disintegrated Tachyzoites

Live Tachyzoites (2×10⁹) were partially purified by gel filtration on a sephadex chromatography column (Amersham Biosciences, Uppsala, Sweden). The collected fractions were sedimented by centrifugation at 1500 g. The live parasites obtained were used for immunization of animals in Group A and for challenge infection. The live parasites were further processed into a disintegrated preparation. The parasites were suspended in 1 ml of 10 mM Tris-hydrochloride containing 2 mM of phenylmethylsulfonylfluoride (Sigma Chemical Co., St. Louis, Mo., USA) and disrupted by ultrasonic treatment (Sonifier 450, Branson Ultrasonic Co., USA) in an ice-bath, and centrifuged at 10,000 g for 20 min at 4° C. The protein content of the recovered pellet was determined by the Micro BCA protein assay method (Pierce, Rockford, USA), and the supernatant aliquoted and cryo-preserved at −80° C. until use as disintegrated experimental vaccines and as antigen for stimulation of whole blood cells in IFN-γ assay.

### Experimental Vaccine Formulations

Various antigen preparations (Rabies, RSV, Influenza, Neospora and Staphylococcus) were formulated with Matrix M or Matrix Q adjuvant. Appropriate amounts of vaccine antigen and Matrix preparation were mixed according to Tables provided in respective Example by simple mixing. The final volume was adjusted with PBS. Experimental vaccine formulations were stored at +2-8° C. prior to use.

### ISCOM Formulations (DiRV, Rabies N-protein, DiRSV)

ISCOMs were prepared according to published standard technology (e.g. EP 0 109 942 B1, EP 0 242 380 B1 and EP 0 180 564 B1). Briefly, 1 mg of recombinant Rabies N-protein, DiRV or

DiRSV were mixed with 1 mg of each cholesterol and Phosphatidyl choline prepared in 20% MEGA-10 and appropriate amount of respective saponin preparation (Fraction A, Fraction C or semipurified “Quit A). The detergent was removed by dialysis. Preparations were filtered through 0.2 mm filter, and the antigen and saponin content was analysed.

Disintegrated Neospora Antigens Formulated with Matrix Q Adjuvant

The live tachyzoites were suspended in PBS and adjusted to 1×10⁸ per calf dose in 3 ml PBS and packed in 5 ml sterile syringes. The live parasites were either used for immunization (Group A) or for challenge infection week 11 (all groups) and were transported in an insulated box at room temperature (RT) to the pens for the administration of the calves within 45 min after harvest from the tissue culture. Disintegrated tachyzoites were mixed to contain 500 μg Neospora antigen supplemented with 750 μg Matrix Q and in a final dose volume of 2 ml (Group B) or suspended in PBS with no adjuvant (Group C).

### Formulation of S.A. Bacterin Vaccines for Example 7a

The experimental vaccine consisted of a Staphylococcus aureus capsular polysaccharide type 5 strain (Reynolds). The organism was kept in frozen stocks at −80° C. and activated in brain heart infusion by overnight incubation at 35° C. One hundred pi of this culture were seeded on Tryptic soy agar added with 2% NaCl and incubated overnight at 37° C. The culture was washed with PBS (pH 7.4), resuspended to achieve a final concentration of 1×10⁹ colony forming units (cfu)/ml and inactivated with 0.5% formalin for 24 hs at 37° C. Sterility of this formulation was evaluated by plating 100 μl on blood agar plates by duplicate. The vaccine was formulated using an alum-based adjuvant system (vaccine 1) and a saponin-based Matrix Q adjuvant. A placebo consisting of sterile saline solution was used as control.

### Formulation of S.A. Bacterin and Lysate Vaccines for Example 7b

(1) Vaccine 1: Staphylococcus aureus capsular polysaccharide type 5 strain (Reynolds). The organism was kept in frozen stocks at −80° C. and activated in brain heart infusion by overnight incubation at 35° C. One hundred μl of this culture were seeded on tryptic soy agar added with 2.5% NaCl and incubated overnight at 35° C. The culture was washed twice with PBS (pH 7.4), resuspended to achieve a final concentration of 1×10⁹ colony forming units (cfu)/mL and inactivated with 0.3% formalin for 24 hs at 35° C. Sterility of this formulation was evaluated by plating 1000 on blood agar plates by duplicate. Vaccine was formulated with Matrix Q at a final concentration of 2 mg/dose.

(2) Vaccine 2 consisted of the same S. aureus capsular polysaccharide type 5 strain grown overnight under the same conditions. Following formalin inactivation, cells were washed twice with PBS, cell density was adjusted to 1×10⁹ cfu/mL and 1 mL of culture was resuspended in 8 mL 50 mM Tris pH 7.5. Lysostaphin (35 U, Sigma Chemical Co., St. Louis, Mo.) in 50 mM Tris pH 7.5/145 mM NaCl was added and incubated 6 h at 37° C. in a water bath with shaking (100 rpm). Reaction was monitored by periodically measuring absorbance at OD₆₀₀ and lysostaphin was inactivated in water bath at 75° C. for 15 min. Mixture was cooled and filtered through 0.45 μm pore size membrane filter to remove intact bacteria. Sterility of this formulation was evaluated by plating 100 μl on blood agar plates by duplicate. This vaccine was formulated with Matrix Q at a final concentration of 2 mg/dose.

(3) A placebo consisting of sterile saline solution plus Matrix Q (2 mg/dose) was used as control.

### Characterization of Matrix and ISCOM Formulations

ISCOM/Matrix preparations are characterized using electron microscopy (negative-staining EM, Dynamic light scattering (OLS) or sucrose-density gradient centrifugation.

Matrix M, (WO 2004004762), wherein compositions comprising ISCOM matrix based on fraction A and C of Quil A in physically different ISCOM matrix complex particles are described and Matrix-Q raw fraction of Quil A (WO 9003184), were supplied by Isconova AB.

### Analyses of Antibody Responses

Antibodies to rabies virus was determined by indirect ELISA using antigens of either N-BV or whole rabiesvirus (Smith J S, Sumner J W and Ruomillat L F. Enzymen immuno assay for rabies antibody in hybridoma cultuture fluids and its application to differentiation of street and laboratory strains of rabies virus. J Clin 1984;19 267-272.)

All mice used in the experiment 1 were female 5-weeks-old females (CDC-ICR). All mice were tested for rabies virus neutralizing (VN) antibodies prior to use; none had rabies virus antibodies or VN antibodies at the time of inocculation. Blood was collected from the mice during the experimental immunized at two weeks interval to determine VN antibodies and anti-N-protein by ELISA.

### ELISA (Rabies)

Indirect ELISA detecting IgG1 and IgG2a antibodies was run according to standard protocols using HRP-labelled anti mouse IgG1 and IgG2a antibody conjugates. The antigen was coated onto Nunc ELISA plates using 50 mM carbonate pH 9.6. The enzyme reaction was visualized using TMB.

### Rabies Virus-Neutralization Antibodies

VN-antibodies in sera from Grey Foxes were analyzed according to OIE. For mouse sera, an alternative in vitro test based on blocking of virus neutralizing monoclonal antibodies was used (“VN-ELISA”). The test was run by EVL, The Netherlands according to Rooijakkers, E., Groen, J., Uittenbogarrd, J., van Herwijnen, J. & Osterhaus, A. (1996). Development and evaluation of alternative testing methods for the in vivo NM potency test used for the quality control of inactivated rabies vaccines. Developments in Biological Standardization 86, 137-145.

### Western Blot (Rabies)

Blood samples (from Ex 1b (3) for ELISA evaluation of antigen specific antibodies taken three weeks after the first and two weeks after the second immunization, were pooled group-wise and analyzed in western blot.

Whole rabies virus preparation (Pitman-Moore strain) and DIRV (TS80 virus preparation) were separated on a 10% gel using standard protocol for SDS-PAGE. Thereafter the proteins were either stained with Coomassie blue or transferred to PVDF membranes for Western blot analysis. The membrane was blocked in PBS containing 0.025% tween-20 and 5% milk powder for 1 hour. Thereafter the membranes were incubated in serum for two hours. Serum from all individuals within each groups were pooled and diluted 1:30 in PBS containing 0.025% tween (PBST). The membranes were washed and incubated with Horseradish peroxidase-conjugated (HRP) anti-mouse IgG antibodies for one hour (Bio-Rad 1:3000 in PBST). After a final wash the blot was developed using chloronaphthol 1-step detection kit (Thermo Scientific). All incubations were performed on a rocket table in room temperature.

ELISA (N. caninum)

Indirect ELISAs to evaluate N. caninum specific serum IgG and subclasses IgG₁ and IgG₂ One μg of solubilized N. caninum tachozoite antigens diluted in 0.06 M carbonate/bicarbonate buffer (pH 9.6) was distributed and adsorbed to each fiat bottom 96-well plates (Polysorp, Nunc). The plates were sealed and incubated overnight at 28° C. and stored at −20° C. until use (Echaide et al., 2002). Once thawed, the plates were incubated at 37° C. for 45 min. The buffer was eliminated and replaced with 200 μl/well of blocking buffer (0.06 M carbonate/bicarbonate with 4% of skimmed milk (Nestle®, Argentina) and incubated at 28° C. for 45 min. The wells were washed four times with 0.01 M PBS-0.05% Tween-20 (PBS-T) plus 4% milk. Negative and strong positive controls (C++) sera, and serum samples were diluted 1/100 in PBS/0.75 M EDTA/EGTA (pH 6.3) plus 4% skimmed milk. One hundred μl of each sample were distributed and incubated on a shaker as above. Conjugate controls (serum free) were included in duplicate. After five washings with PBS-T, two alternative procedures were followed. To evaluate total bovine IgG, wells were filled with 100 μl of 1/1000 dilution of the anti-bovine IgG polyclonal antibody conjugated to peroxidase (Sigma, USA) and incubated on a shaker for 60 min. After four washings, 100 μl of 3% H₂O₂/0.04 M ABTS (2,2′-azino-bis 3-ethylbenzothiazoline-6-sulphonic acid) (Sigma, St. Louis, USA) were added as substrate/chromogen. A kinetic reading (Multiskan RC, Labsystems, Helsinki, Finland) was determined at an optical density of 405 nm (OD₄₀₅) when N. caninum C++ reached 1.0±25%. The OD₄₀₅ of sera were expressed as percentage of positivity (PP) related to C++ according to the formula: PP=(mean serum OD₄₀₅×100)/mean C++ OD₄₀₅. The cutoff point used was ≧25 PP.

To assess the IgG₁/IgG₂ rate, wells were filled with 100 μl of 1/100 dilution of anti-bovine IgG₁ or IgG₂ mAbs (Serotec™, Oxford, UK) in PBS-T and incubated during 30 min. Each serum was simultaneously evaluated with both mAbs in the same plate. After four washings 100 μl of anti-mouse IgG mAb conjugated to peroxidase (Jackson®), diluted 1/1000 was added and incubated on a shaker for 30 min. After four washings, 100 μl of 3% H₂O₂/0.04 M ABTS were added. For IgG₁ and IgG₂, a kinetic reading was determined at an OD₄₀₅ when N. caninum C++with anti-IgG₁ reached 1.0±25%. Data were expressed as a ratio of OD values for IgG₁/OD value for IgG₂.

ELISA (Staphylococcus aureus)

Briefly, flat-bottomed 96-well microtitre plates were coated overnight at 4° C. with solution of antigens (Bacterin or Lysate) in PBS (pH 7.2) containing 1 μg/well. Between each step, plates were washed five times with 0.05% Tween 20. The coated plates were first incubated for 1 h at 37° C. with PBS with low fat milk 5% free from antibodies. The 1/500 dilutions in PBS of heifer test sera was distributed in duplicate and incubated for 1 h at 37° C., followed by 1/2000 dilution of peroxidase-conjugated goat anti-cow IgG (H+L). After incubation enzyme substrate was added. After 10 min at room temperature, the reaction was stopped by the addition of 2 N H₂SO₄. The absorbance was read at 450 nm. Antibody levels were expressed as ELISA index, calculated by dividing the absorbance reading of the test serum by the absorbance reading of a pool of high-titered immune mouse serum.

### Analysis of Cellular Responses

In vitro Restimulation of Splenocytes (Rabies)

Female mice (BALB/c) 10-12 weeks old, were immunized twice according to Table 3. Approximately two weeks after (booster) the second immunization the mice were sacrificed and spleen removed and a single cell suspension prepared. The splenocytes (5×10⁵ cells/well in 200 μl) were plated in 96-well plates and were stimulated for 72 hours with either WCRV (2.5 μg/ml), or purified rabies N-protein (0.1 μg/ml), or Con A as positive control (2.5 μg/ml) or sterile RPMI-medium as negative control,. Supernatants were collected and stored in −70° C. until analysis with cytometric bead array (CBA, BD Bioscience), in order to determine cytokine concentrations. Cytokines analyzed were, for T cells in general; interleukin (IL)-2 and for Th1 cells; interferon (IFN)-gamma and for Th2 cells; IL-4 and IL-5. Data collection and analysis were performed on a FACSCanto flow cytometer.

### Cell Proliferation Assay

Splenocytes (2.5×10⁵ cells/well in 100 ml) were stimulated with either whole inactivated rabies virus (Abs, 2.5 mg/ml, 0.5 mg/ml and 0.1 mg/ml), or rabies N protein (2.5 mg/ml, 0.5 mg/ml and 0.1 mg/ml), or rabies G protein (2.5 mg/ml, 0.5 mg/ml and 0.1 mg/ml), or Concanavalin A (Con A) as positive control (2.5 mg/ml) or sterile RPMI culture medium as negative control, for 42 hours. Cellular proliferation i.e. DNA synthesis, was measured using a BrdU-ELISA assay (colorimetric) according to the manufacturer's protocol (Roche Diagnostics GmbH, Germany). Absorbance was measured on a spectrophotometer at the test wavelength 370 nm and the reference wavelength 492 nm.

### Assessment of Cytokine Secretion, Balance of the Th1/Th2 Immune Response

Further, secreted cytokines were measured in in vitro in culture medium after antigen stimulation of splenocytes from in vivo immunized mice. Splenocytes (5×10⁵ cells/well in 200 ml) were plated in 96-well plates and were stimulated with either whole inactivated rabies virus (2.5 mg/ml, 0.5 mg/ml and 0.1 mg/ml), or rabies N protein (2.5 mg/ml, 0.5 mg/ml and 0.1 mg/ml), or rabies G protein (2.5 mg/ml, 0.5 mg/ml and 0.1 mg/ml), or Con A as positive control (2.5 mg/ml) or sterile RPMI-medium as negative control, for 72 hours. Supernatants were collected and stored in −70° C. until analysis with cytometric bead array (CBA), in order to determine cytokine concentrations. Cytokines analyzed were, for T cells in general; interleukin (IL)-2 and for Th1 cells; interferon (IFN)-g and for Th2 cells; IL-4 and IL-5. Data collection and analysis were performed on a FACSCanto flow cytometer.

Assessment of N. caninum-Specific IFN-γ Responses

Immune stimulation was performed as mentioned Serrano-Martinez at al., (2007). Briefly, 0.9 ml of heparinised whole blood was dispensed into each of two wells of 24-well tissue culture plates (Cellstar Greiner, USA) and cultured with 0.1 ml of PBS (unstimulated control), concanavalin A (Con-A, Sigma, St. Louis, USA) at 10 μg/ml to ensure cellular ability to respond to stimulation and secrete IFN-γ, and with disintegrated antigen from the N. caninum NC-1 strain (1 μg/ml). Heparinised whole blood samples were incubated in a 5% CO₂ atmosphere for 16 h at 37° C. Plasma was harvested from each well and frozen at −20° C. until testing. To assess IFN-γ production, plasma samples were tested using a commercial ELISA kit (Bovigam IFN-γ kit, CSL, Australia), according to the manufacturer's recommendations.

### RSV Vaccination and Challenge in Cotton Rats

Rats were immunized twice at 3 week intervals (on days 0 and 21) with the DiRSV, prepared as described above, in 1 μg or 5 μg doses adjuvanted with Matrix-M (24 μg/dose) in a total volume of 200 μL (Table 5). The vaccine or controls (placebo or infectious virus) were injected intramuscularly (i.m.) in volumes of 100 μLs in each leg of the rat (see table 1 below). Animals in all groups except in group 5 were challenged infected on day 46 under lightly anaesthetize (Isoflurane) with a dose if 10⁵ PFU RSV strain Tracy in 100 μL.

Five groups of 6 animals are included in the example.

Bleedings for serum taken at days 0, 21, 46 and 50 were analyzed for anti-RSV neutralizing antibodies. The rats were bled and sacrificed for isolation of virus in the upper respiratory tract (URT) and the lungs virus on day 50.

Lunglavage (lung & URT), Virus isolation & (PFU) and Virus neutralization was performed according to standard and published procedures (for references see, e.g., Hu et al., Clin Exp Immunol 113 p.235, 1998).

### Neospora Immunization and Challenge

Group A were inoculated intravenously day 0 with 10⁸ live tachzoites while animals in Groups B through E received 2 subcutaneous inoculations laterally on the neck on day 0 and the second dose was given on the other side 4 weeks later. All calves were challenged with 1×10⁸ tachyzoites of NC-1 strain by intravenous inoculation at week 11. Fifteen five months old Aberdeen Angus calves, were randomly distributed into 5 experimental groups with three animals per group were observed daily throughout the experimental period.

Ex vivo Human DC Model for Evaluation of Matrix Adjuvant

### Cells Culture and Stimulation

30 ml blood was collected from each one of 5 adult volunteers. PBMC were separated by a density gradient (Lymphoprep; Nycomed), counted in a Neubauer chamber, and the viability was assessed by Trypan blue dye exclusion. Monocytes of 90-99% purity were obtained by negative depletion using magnetic separation according to manufactures recommendation (Monocyte isolation Kittll, Miltenyl Biotec Inc.). The monocytes were cultured at a concentration of 10⁶/ml of AIM-V medium plus 10% fetal calf serum, 50 ng/mL GM-CSF and 50 ng/mL IL-4 at 37° C. with 5% CO₂. Immature dendritic cells (iDCs) were obtained after 5 days of culture. Immature DCs were further cultured for 24 h in the presence of either; medium, LPS (1 μg/mL), Matrix-A at 200 μg/mL, 100 μg/mL, 10 μg/mL, Matrix-C at 10 μg/mL, 1 μg/mL, 0.1 μg/mL, Matrix M at 100 μg/mL, 10 μg/mL, 1 μg/mL. Thereafter, the DCs were analyzed for the following surface proteins: CD11c, CD14, CD83, CD86 and HLADR using monoclonal antibodies for identification and for quantification by flow cytometry (FACscan Beckton Dickinson, San Jose Calif., US).

### EXAMPLE 1

### Qualitative Improvement of Traditional Rabies Vaccines by Matrix M Adjuvant Formulation and Virus Particle Disintegration

Rabies infection is a zoonotic fatal infection of warm-blooded animals. The only modus operandi for protection available for animals and man is vaccination; prophylactic to prevent disease or after expected virus exposure as post-exposure treatment together with hyper-immune serum. Post exposure treatment of animals after suspected rabies virus exposure is not allowed or practised.

Rabies vaccines used for man and animals are similar, differing in that adjuvants i.e. Alum adjuvants (Al(OH)₃ or AlPO₃) are used in most animal rabies vaccines while no adjuvants are used in man. The present vaccines are conventional, they induce predominantly a TH2 type of response and have not faced development for the last 50 years. For registration and efficacy evaluations (e.g. batch release) of rabies vaccines, only virus neutralization antibody testing according to the NIH test is required and practiced. Example 1 A-D explore and demonstrate the beneficial effects of including Matrix M as adjuvant in rabies virus vaccines to induce broader protective immune responses (1a) including also internal antigens e.g., the rabies N-protein; (1b) improving magnitude and quality of antibody responses in mice (1), in Grey Fox (2), as demonstrated by Western blot analysis (3) and by improved performance of two commercially available WRV vaccines (4).

### EXAMPLE 1A

### ISCOM Formulation Triggers Internal Rabies N-Protein to Induce Protective Immunity

This example was designed to explore whether an internal virus protein adjuvanted with a potent adjuvant such as Matrix M can induce immune protection. A recombinant Rabies virus nucleoprotein (N-protein) produced in insect cells transformed by Bacculovirus (see M & M) was used excluding the presence of other rabies virus components. The rabies N-protein formulated as ISCOMs (see M&M section) vaccine was administered (SC, IM and IP) to mice in 1 and 5 pg doses and was compared to a 25 μg dose (SC, IM) of the non-adjuvanted N-protein vaccine (see tables 1.1 and 1.2 for experimental setup and results). The experimental vaccines were administered days 0 and 7 for a primary immunization, being the standard for testing rabies vaccines according to the NIH test. The 25 μg dose of the non-adjuvanted N-protein vaccine was selected since preliminary experiments indicated that such a dose was required to detect immune protection according to the NIH test. The N-protein ISCOMs were also immunogenicity tested in Balb/c mice (see Table 1.3). Blood samples for sera were taken at days 14, 29, 45, 58 and 72. The sera were tested in ELISA against the recombinant Rabies N-protein and Rabies virus.

TABLE 1.1
Experimental setup and Protection to challenge¹ infection. Mice
immunized² with 25 μg of non-adjuvanted Rabies N-protein.

| Mode of | Vaccine group | Control group |
|---|---|---|
| administration | no protected/total | no protected/total |
| IM | 3/10 | 0/10 |
| SC | 4/10 | 0/10 |

¹Footpad challenge with street rabies virus at day 60.
²CDC-ICR mice were vaccinated with 25 μg of non-adjuvanted Rabies N-protein at days 0, 7 and 28 according to NIH test for Rabies vaccines

TABLE 1.2
Experimental set up. Protection to challenge infection
experiment in mice immunized with 1 and 5 μg of
ISCOM formulated Rabies N-protein ISCOM.

| | Challenge¹ | | | | | |
|---|---|---|---|---|---|---|
| Mode of | 14 days after 1^{st} immunization² | | | 60 days after 1^{st} immunization² | | |
| adminis- | Vaccine dose | | | | | |
| tration | 1 mg | 5 mg | Control | 1 mg | 5 mg | Control |
| IM | 20/20 | 19/20 | 0/10 | 8/10 | 10/10 | 0/10 |
| SC | 19/20 | 20/20 | 0/10 | 6/10 | 10/10 | 0/10 |
| IP | 17/20 | 20/20 | 0/10 | 6/10 | 9/10 | 0/10 |

¹Footpad challenge with street rabies virus
²Mice were vaccinated at days 0, 7 and 28 according to NIH test for Rabies vaccines

TABLE 1.3
Non-neutralizing anti-rabies antibodies measured by ELISA in sera
from mice Test antigen

| | Days post 1^{st} immunization¹ | | | | |
|---|---|---|---|---|---|
| | 14 | 29 | 43 | 58 | 72 |
| | Reciprocal antibody titer¹ | | | | |
| N-protein | 50 | 50 | 2 200 | 1 800 | 1 500 |
| WRV | 1 500 | 2 000 | 48 500 | 32 100 | 24 500 |

¹Balb/c were vaccinated with 1 □vaccinated² formulated Rabies N-protein at days 0, 7 and 28.
The N-²ISCOMs are identical to those used in the challenge study presented in Table 1.2 above.

### Results

A primary immunization schedule (day 0 and 7) with 25 mg of non-adjuvanted N-protein induced poor protection after SC or 1M immunization (Table 1.1). In contrast, identical primary immunization with 1 or 5 μg N-protein ISCOM induced full protection (20/20 mice) already 14 days after a primary IM immunization (Table 1.2). The SC and IP routes induced a slightly lower level of protection, 19/20 and 17/20 respectively. After booster, 5 μg N-protein ISCOM induced almost full protection to challenge at day 60 after first administration regardless of route of administration (Table 1.2). With a dose of 1 μg N-protein ISCOM, the protection was about 60-80% at the challenge day 60 (Table 1.2).

None of the non-immunized mice survived the challenge infection (Table 1.1).

No “classical” virus neutralizing (VN) antibodies were detected (not shown) while high levels of antibodies against the NP were detected in ELISA (Table 1.3).

### Discussion and Conclusion

This example shows that protective immunity can be induced with Rabies virus N-protein provided that a potent adjuvant is used. Thus, additional protective mechanism(s) (besides virus-neutralizing antibodies to the G-protein) was activated by the adjuvant. The protective immunity evoked by the N-protein ISCOM is not due to protective antibody responses since the antibody titers to N-protein and WRV were low at the time for challenge. The protective immunity must be dependent on cell-mediated immunity, which most likely includes Th1 and CTL responses. The fact that protection was induced rapidly is particularly important for a post-exposure vaccine effect. This example demonstrates that a fast protective immune response can be induced by the internal rabies virus N-protein after ISCOM formulation. Since the NIH test protocol for releasing a rabies vaccine batch was used for immunization, the optimal immunization protocol for the ISCOM adjuvanted experimental vaccine was not applied i.e. a first dose day 0 and a boost week 4 to 6. The possibility to use the N-protein is likely to broaden the protective immunity e.g. to also include protection against (B)bat Lyssa viruses e.g. the Duvenhagen strain.

The results are novel in view of; (I) no rabies G-protein was present, (II) the dose (1-5 μg) was low (compared to 25 μg of non-adjuvanted antigen), (III) the time lapse of one week after completed priming is short. Moreover, a long-lasting immune response was induced.

### EXAMPLE 1b

### Matrix M Improves Rabies Virus Vaccine Formulations Measured by Magnitude and Quality of Antibody Responses

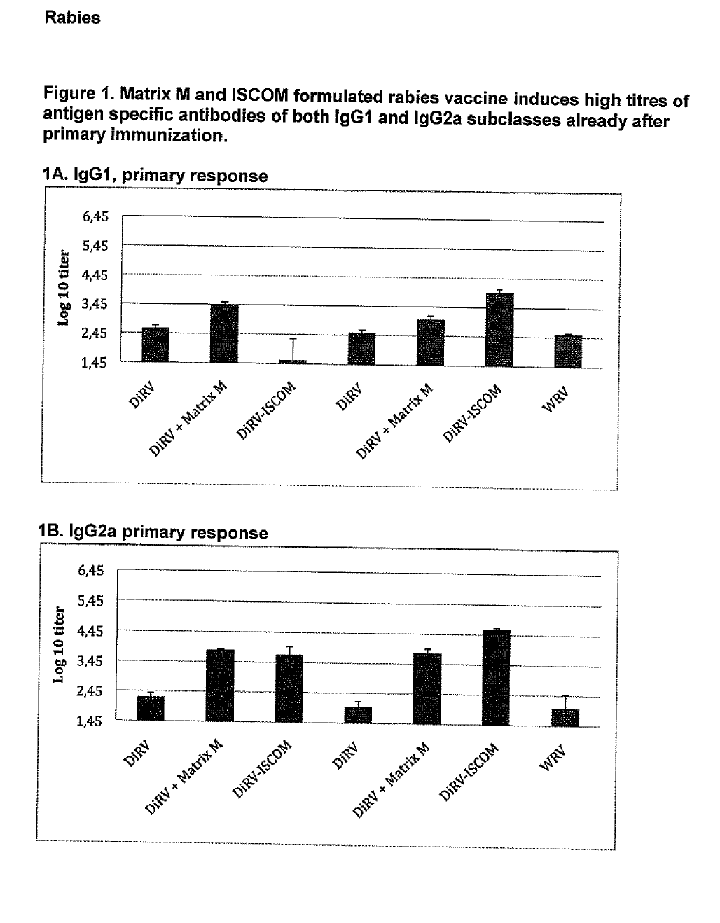

(1) This example was carried out in mice to explore the beneficial effect of Matrix M on different Rabies-virus antigen formulations as described in Table 2:1. The Rabies virus antigens WRV (whole virus), DiRV (disintegrated rabies virus) were formulated with or without Matrix M or formulated as ISCOMs. The results are shown in FIGS. 1-3. Balbfc mice were vaccinated s.c. in the neck with the different formulations in Table 2.1.

TABLE 2.1
Immunization with WRV or vaccine formulations adjuvanted
with Matrix M or formulated in ISCOM

| | | | | | Immuni- | |
|---|---|---|---|---|---|---|
| | | | | | zation/serum | |
| | Antigen | | Dose | No | samples | Study |
| Group | form | Adjuvant | (IU) | mice | (weeks) | parameter |
| 1 | DiRV^{A} | — | 0.03 | 8 | 0, 4/3, 6 | IgG1, IgG2a, |
| | | | | | | VN-ab |
| 2 | DiRV^{A} | Matrix M¹ | 0.03 | 8 | 0, 4/3, 6 | IgG1, IgG2a, |
| | | | | | | VN-ab |
| 3 | DiRV^{A} | ISCOM² | 0.03 | 8 | 0, 4/3, 6 | IgG1, IgG2a, |
| | | | | | | VN-ab |
| 4 | DiRV^{B} | — | 0.02 | 8 | 0, 4/3, 6 | IgG1, IgG2a, |
| | | | | | | VN-ab |
| 5 | DiRV^{B} | Matrix M¹ | 0.02 | 8 | 0, 4/3, 6 | IgG1, IgG2a, |
| | | | | | | VN-ab |
| 6 | DiRV^{B} | ISCOM² | 0.02 | 8 | 0, 4/3, 6 | IgG1, IgG2a, |
| | | | | | | VN-ab |
| 7 | WRV^{P} | — | 0.1 | 8 | 0, 4/3, 6 | IgG1, IgG2a, |
| | | | | | | VN-ab |

^{A}TS80 strain
^{B}ERA strain
^{P}Pitman Moore strain (commercial whole virus vaccine)
¹Matrix M dose (7.5 mg)
²Saponin content and composition identical to Matrix M

### Results

1^{st} immunization in mice (FIGS. 1A and B). The antibody responses (IgG1 and IgG2a) to Rabies virus antigens were measured by ELISA (see M&M). The highest antibody level after one immunization was induced by a DiRV formulated as ISCOM² vaccine inducing approximately 50 fold higher IgG1 titres than the non-adjuvanted WRV corresponding to a conventional rabies virus vaccine. The two Matrix M adjuvanted preparations induced about 5-fold higher antibody titers than the WRV. Even more impressive was the IgG2a antibody response induced by ISCOM and Matrix M adjuvanted formulations, reaching ≈100-fold higher antibody levels than those of WRV. Non-adjuvanted DiRV preparations induced moderate levels of IgG1 antibody and comparatively low levels of IgG2a antibodies.

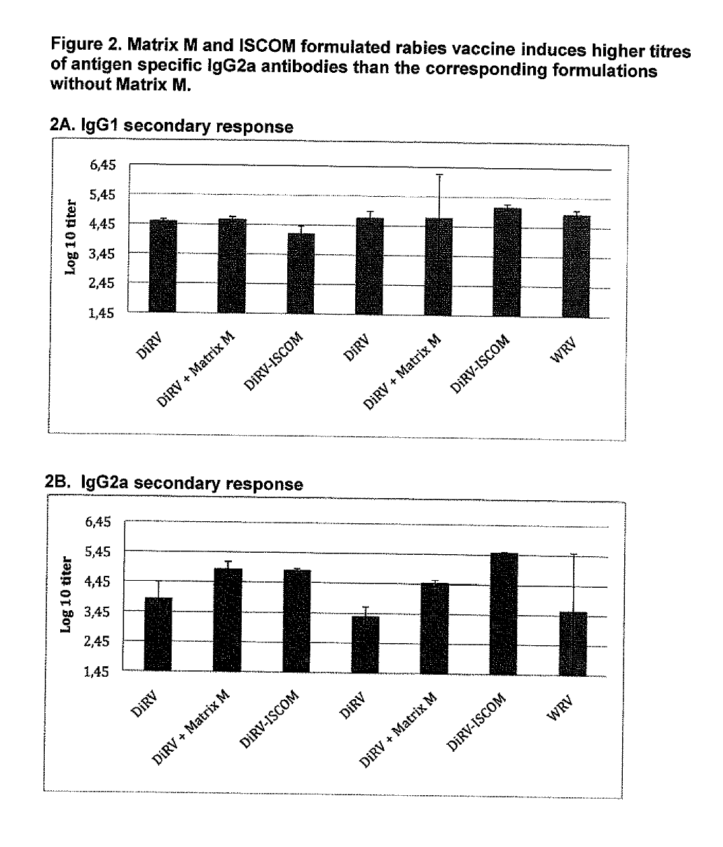

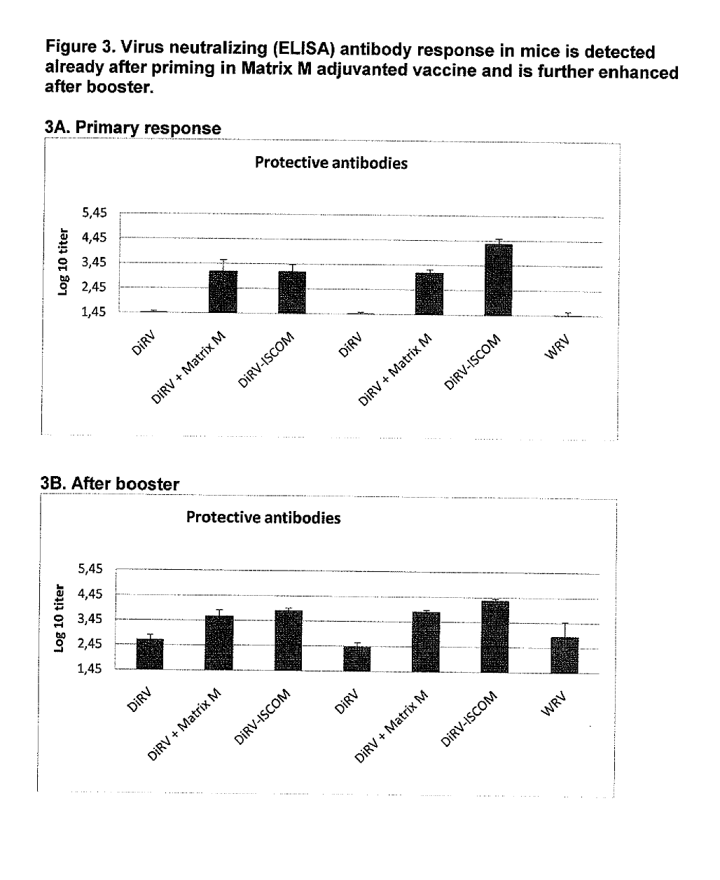

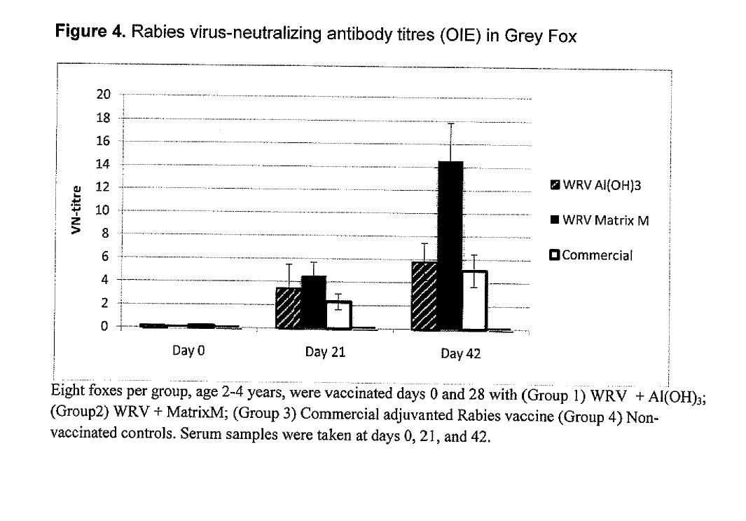

2^{nd} immunization in mice (FIGS. 2A and B). All formulations induced similar levels of IgG1 antibodies whereas ISCOM and Matrix M adjuvanted formulations induced considerably higher levels of IgG2a antibodies (1-2 logs higher than non-adjuvanted WRV or non-adjuvanted DiRV. Virus neutralizing antibody response (“VN-ELISA”) in mice (FIGS. 3A and B). The VN antibody response essentially followed that of the IgG2a response. The ISCOM and Matrix M adjuvanted formulations induced high levels of VN response already after the first immunization. Neither WRV nor non-adjuvanted DiRV induced VN antibodies after 1^{st} immunization. After booster, ISCOM and Matrix M adjuvanted preparations induced VN titers≧1 log higher than WRV. (2) A second experiment was carried out in Grey Fox to corroborate the mouse data in a relevant target animal species. Grey Foxes were immunized with WRV (whole rabies virus) preparation alone or adjuvanted with Al(OH)₃ and Matrix M respectively (see table 2.2). The results of the study are shown in FIG. 4.

TABLE 2.2
Immunization of Grey Fox with different experimental Matrix M
adjuvanted rabies virus vaccines

| Group | No of foxes | Antigen | Adjuvant |
|---|---|---|---|
| 1 | 8 | WRV | Al(OH)₃ |
| 2 | 8 | WRV | Matrix M |

| | | |
|---|---|---|
| 3 | 8 | Commercial AlPOH₃ adjuvanted WRV |
| 4 | 2 | Non-immunized animals |

The animals were immunized at weeks 0 and 4, serum samples were taken at weeks 3 and 6.

### Results

VN response (according to OIE) in Grey Fox (FIG. 4). Grey Foxes immunized with Matrix M adjuvanted WRV induced the highest levels of VN antibodies after the first immunization but most prominently after booster, where the VN-titers for the Matrix-M adjuvanted group was 16 compared to ≦6 for Commercial (Group 3) and Al(OH)₃ (Group 1) reference vaccines. The results in Grey Fox support those measured in mice.

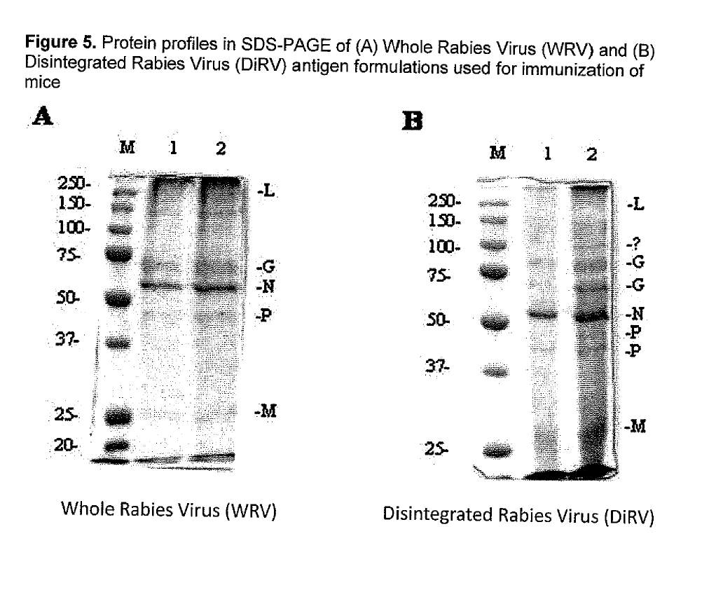

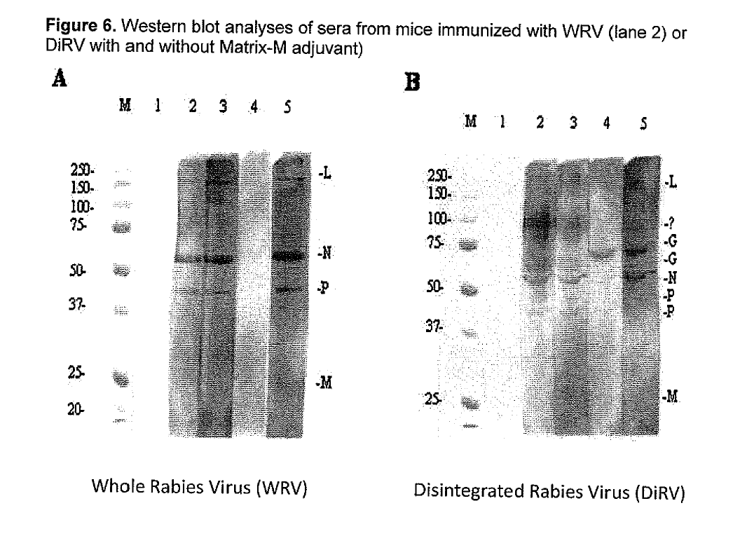

(3) In order to assess the influence of the antigen formulation, i.e., WRV or DiRV (i.e. whole vs. disintegrated virus antigen preparations) Western-blot analysis were run on sera from mice immunized with WRV or DiRV with or without Matrix M. The results are shown in FIGS. 5 and 6. Since the WRV is of Pitman More strain and DiRV of the TS80 strain the sera were tested against SOS-separated proteins from both strains (A and B respectively).

### Results, SOS-PAGE and Western Blot Analysis

### Protein Profile Analyzed by Polyacryl Gel Electrophoresis (PAGE)

Coomassie stained of WRV (FIG. 5A) and DiRV (FIG. 5B) virus preparations revealed the same pattern i.e. all five viral proteins (L-, G-, N-, P-, and M-protein) are detected in both viral strains (FIGS. 5A and B).

### Western Blots Analysis

Serum from mice immunized with DiRV without Matrix M formulation (lanes 2 in FIGS. 6A and B) detected more protein bands than the sera from mice immunized with WRV without Matrix M formulation (lanes 4 in FIGS. 6A and B). Thus, the disintegrated virus exposed more proteins that stimulated specific antibody formation e.g. N and P proteins. In contrast, no N or P, being internal proteins, were detected from mice immunized with non-adjuvanted whole virions. Sera from mice immunized with DiRV adjuvanted with the Matrix M formulation (lane 3 in FIG. 6A) detected no additional proteins i.e. similar proteins were detected in WB as with sera of mice immunized with whole virions adjuvanted with Matrix M in (lane 5 in FIG. 6A). Lane 3 in FIG. 6B blotted with serum from mice immunized with DiRV adjuvanted with Matrix M revealed similar pattern of bands as sera from mice immunized with adjuvanted WRV (whole virions) (lane 5 FIG. 6B). These results indicate that non-adjuvanted DiRV reveals more antigens stimulating to antibody responses than whole virions. In contrast that difference could not be detected after that the virus antigens had been supplemented with the Matrix formulation explained by the potent adjuvant effect.

Interestingly, in order to broaden the immune response to internal antigens, the rabies virus can be disintegrated. However, it is essential that a potent adjuvant like Matrix-M is used to stimulate the immune system, resulting in induction of immune responses to the revealed antigens. The likely reason is that Matrix M enhances immunogenicity of small and low amount) of rabies virus antigens. To stimulate induction of immune responses also to minor components of a vaccine(s) is an important property of an adjuvant; to induce immune protection as well as for antigen saving in vaccines.

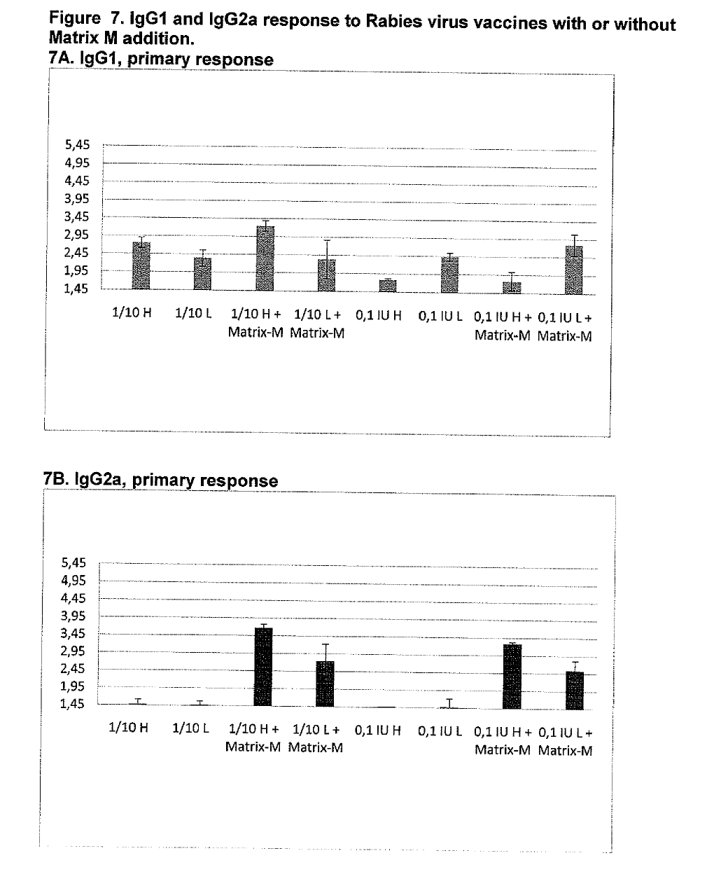

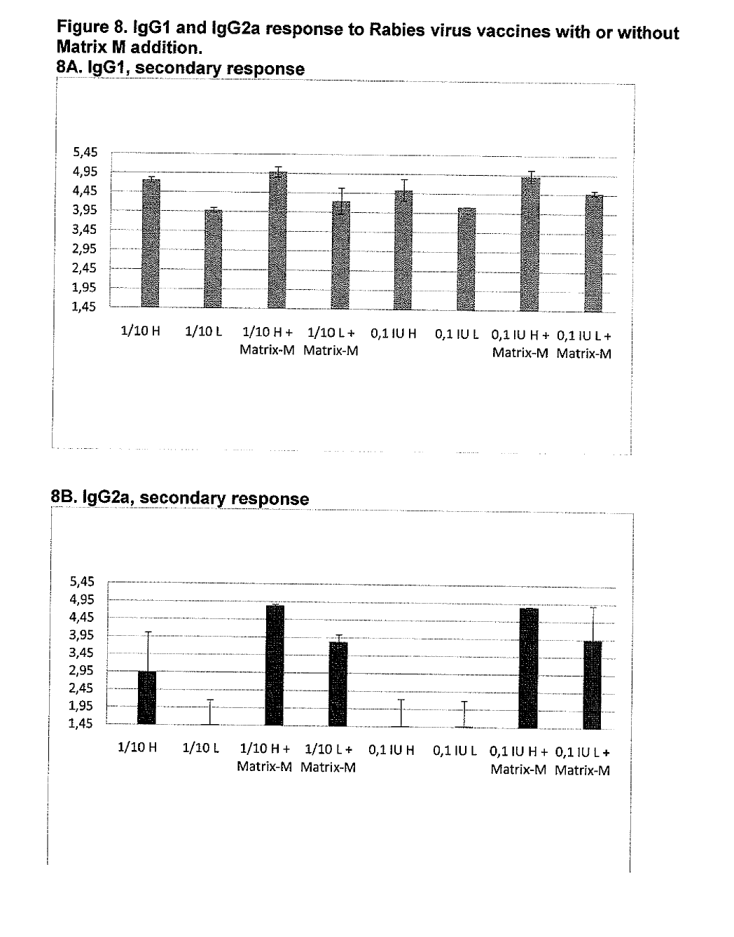

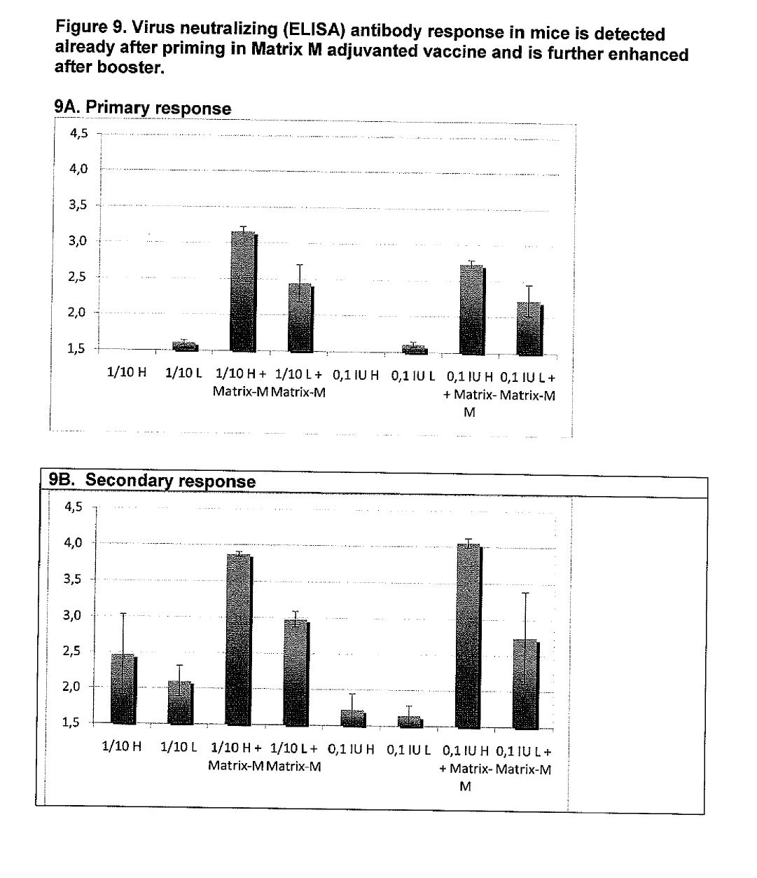

(4) In this experiment two different commercially available vaccines canine products were potentiated with Matrix M. One of the vaccines contained high (HIGH) amounts of Rabies antigen measured as IU (standard international units) and the other product contained a low (LOW) amount of antigen measured as IU. The vaccines were dosed in two ways; 1/10 of a dog dose or 1/10 IU as listed in Table 2.3. The results are shown in FIGS. 7-9.

TABLE 2.3
Comparison of two Canine Rabies vaccine preparations
adjuvanted with Matrix M.

| Group | No of mice | Vaccine | Dose | Addition |
|---|---|---|---|---|
| 1 | 8 | HIGH | 1/10 dose | — |
| 2 | 8 | LOW | 1/10 dose | — |
| 3 | 8 | HIGH | 1/10 dose | Matrix M |
| 4 | 8 | LOW | 1/10 dose | Matrix M |
| 5 | 8 | HIGH | 1/10 IU | — |
| 6 | 8 | LOW | 1/10 IU | — |
| 7 | 8 | HIGH | 1/10 IU | Matrix M |
| 8 | 8 | LOW | 1/10 IU | Matrix M |

HIGH-vaccine containing 3 IU/dose
LOW-vaccine containing 1 IU/dose

### Results

Matrix M strongly potentiates the antibody response in mice to both rabies vaccines. (FIGS. 7-9). In particular the IgG2a and the VN responses, (measured by blocking ELISA) were enhanced. The antibody responses developed also faster after addition of Matrix M. Compared to the corresponding preparations without Matrix M both Matrix M adjuvanted vaccines induced high IgG2a and VN titers already after the 1^{st} administration after the 2^{nd} immunization. Thus, a dos-sparing potential was demonstrated.

### Discussion and Conclusion

Matrix M adjuvanted rabies vaccines induced high levels of antibody already after one (primary) is immunization, including functional protective Virus Neutralizing (VN) antibodies. A fast functional protective immune response is more important for a rabies virus than for other vaccines in view of the fact that it is used post-exposure to inhibit a suspected virus infection. Moreover, the combined immune protection exerted by two arms of the immune system has added immune protective effect. Moreover, as shown in example 1a the inclusion of immune response to the N protein of rabies virus broaden the immunity to rapidly induce a protective immune response.

### EXAMPLE 1c

### Matrix M Adjuvanted Rabies Virus Formulations Induce T-helper 1 (TH1) and TH2 Responses in Mouse

In the present example, T cell responses are analyzed in mice after immunization with whole rabies virus (WRV) or disintegrated rabies virus (DiRV) with and without addition of Matrix M. IL-2 production is indicative for strong T cell responses, IFN-γ production is produced by TH1 type T cells and IL-4 and IL-5 are produced by TH2 type T cells. A combination of IL-2 and IFN-_{y} production are essential Th1 components for combating virus infections.

Mice were vaccinated twice with different formulations as indicated in Table 3. Two weeks after the second immunization, spleen cells were re-stimulated with N-protein or WRV. The supernatants of the stimulated spleen cells were screened for production of IL-1, IFN-γ, IL-4 and IL-5.

TABLE 3
T cell responses in mice vaccinated with different experimental
Rabies virus formulations

| | Number | Treatment (mice | | |
|---|---|---|---|---|
| | of | were vaccinated | In vitro | |
| Group | animals | twice) | stimulation | Study parameter |
| 1 | 7 | PBS pH 7.2 | N-protein | IL-2, IFN-γ, IL-4, IL-5 |
| | | (negative control) | WRV | |
| 2 | 8 | DiRV (0.1 U) | N-protein | IL-2, IFN-γ, IL-4, IL-5 |
| | | | WRV | |
| 3 | 8 | DiRV (0.1 U) + | N-protein | IL-2, IFN-γ, IL-4, IL-5 |
| | | Matrix M (7.5 μg) | WRV | |
| 4 | 7 | WRV (0.1 IU) | N-protein | IL-2, IFN-γ, IL-4, IL-5 |
| | | | WRV | |
| 5 | 7 | WRV (0.1 IU) + | N-protein | IL-2, IFN-γ, IL-4, IL-5 |
| | | Matrix M (7.5 μg) | WRV | |

Disintegrated rabies virus (DiRV) of TS80 strain as described in experiment 1b. Whole Rabies Virus vaccine preparation conatining the Pasteur RIV strain (WRV). The experimental vaccines were diluted in PBS and were given as 200 μl injections s.c. in the neck.

### Results

T cell responses to experimental Rabies virus vaccine formulations were measured as profile of the cytokine production after re-stimulation in vitro of spleen cells.

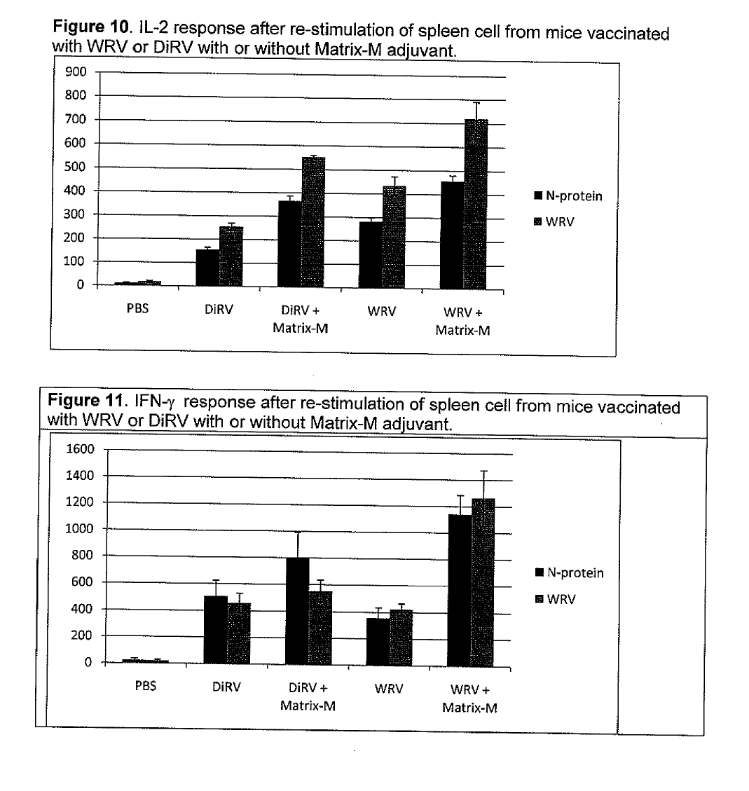

I-2 production after re-stimulation with rabies virus N-protein. In FIG. 10, it is clearly demonstrated that mice immunized with Matrix M adjuvanted N-DiRV or WRV responded with enhanced production of IL-2 detected after re-stimulation of spleen cells in vitro with rabies N-protein, demonstrating that a strong T cell response to the Rabies N-protein was induced by both DiRV and WRV formulated with Matrix.

IL-2 production after re-stimulation with whole rabies virus (WRV): Similar results, measured as IL-2 production, was obtained after re-stimulation of the spleen cells with WRV (FIG. 10).

The IFN-γ production after re-stimulation with rabies virus N-protein: Similarly to the IL-2 production, it is clearly demonstrated that Matrix M adjuvant enhanced DiRV and WRV to induce the production of IFN-γ (FIG. 11.) detected after re-stimulation of the spleen cells with rabies N-protein.

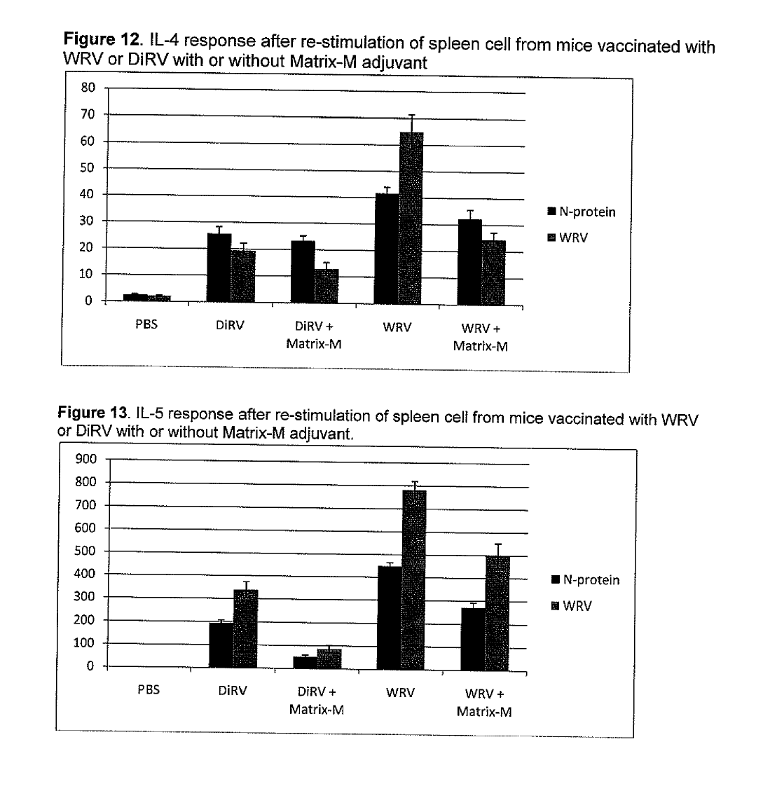

Production of Th2 cytokines, IL-4 and IL-5, after re-stimulation with rabies virus N-protein or whole rabies virus (WRV). Matrix M did not enhance the IL-4 or IL-5 production after re-stimulation with Rabies virus N-protein or WRV (FIGS. 12 and 13). Contrary, the mice vaccinated with non-adjuvanted DiRV or WRV rather produced somewhat higher levels of IL-4 and IL-5 after re-stimulation with N-protein or WRV than mice vaccinated with the Matrix M adjuvanted formulations.

### Discussion and Conclusion

Matrix M enhances the cell mediated TH1 immune responses to both WRV and DiRV virus formulations, which is reflected by the Th1/Th2 ratio. The cellular TH2 type response was not enhanced by the Matrix M adjuvant even though a potentiation of serum IgG1 responses was noted (see Example 1b (1). Both WRV and DiRV virus formulations evoked high levels of antibody and cell mediated immune responses well above the levels of those induced by rabies vaccines available today and above expectations. Both arms of the immune response are essential components to optimize immune protection both for prophylaxis and for post exposure immune treatment of rabies virus infection.

In conclusion: It is well established that the Th1 response is promoting immunity to virus infection. Thus, both the experimental Matrix M formulations, with WRV and DiRV, induced superior immune responses to rabies virus antigen than the immune responses induced by rabies virus vaccines available on the market today.

### EXAMPLE 2

This experiment was designed to explore the enhancing effect of Matrix M on a commercial disintegrated non-adjuvanted influenza vaccine (D-FLU) in a mouse model. Considerations were taken to level of immune response, quality and antigen sparing. Moreover, the duration of the immune response is an additional important factor.

Antigens and experimental layout A commercial trivalent disintegrated influenza (D-Flu=disintegrated Flu) vaccine was used as antigen source. The experimental layout in mice is presented in Table 4. The mouse antigen dose was reduced by a factor of 1:30 of a human dose being standard for testing human vaccines in mice. Additionally, a further reduction of the dose to 1:300 was used to explore antigen sparing effect with the Matrix M adjuvant.

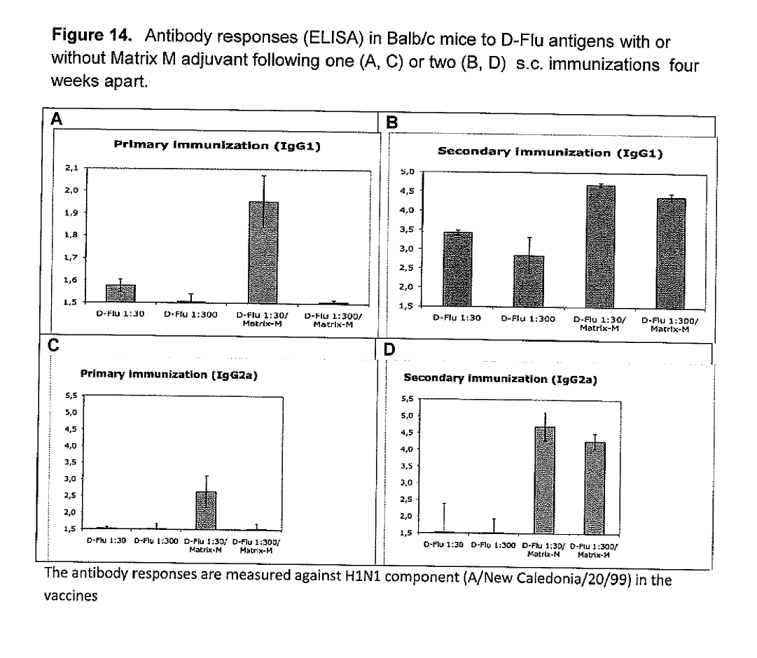

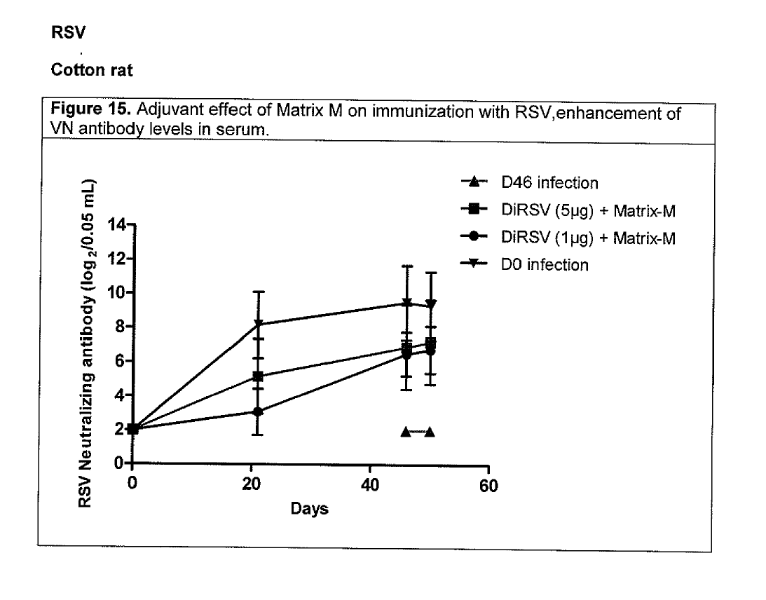

Mice, Immunization and sampling: 18 g female Balb/c mice were immunized as indicated in Table 4. The mice were immunized s.c. at weeks 0 and 4. Blood samples for testing were taken at weeks 3 and 6. The antigen specific antibody responses in IgG1 and IgG2a subclasses at weeks 3 and 6 are shown in FIG. 14(A-D).

TABLE 4
Experimental setup for immunization of Balb/c mice immunized
s.c. with a commercial influenza vaccine and the same vaccine
formulated with Matrix M.

| | | | No of |
|---|---|---|---|
| Group | Antigen | Adjuvant | animals |
| 1 | D-Flu (1:30) | no adjuvant | 8 |
| | 1.5 ug/dose | | |
| | i.e., 0.5 ug per Flu/strain | | |
| 2 | D-Flu (1:300) | no adjuvant | 8 |
| | 0.15 ug/dose | | |
| | i.e., 0.05 ug per Flu/strain | | |
| 3 | D-Flu (1:30) | 10 ug | 8 |
| | 1.5 ug/dose | Matrix M * | |
| | i.e., 0.5 ug per Flu/strain | | |
| 4 | D-Flu (1:300) | 10 ug | 8 |
| | 0.15 ug/dose | Matrix M * | |
| | i.e., 0.05 ug per Flu/strain | | |

* Matrix M formulation consists of a mixture of 90% ug Matrix A and 10% Matrix C.

### Results

After one s.c. immunization, mice immunized with the 30 fold reduced human vaccine dose (1.5 μg) formulated with the Matrix M responded with clear cut IgG1 and IgG2a antibody responses. Mice immunized with the commercial D-FLU vaccine formulation alone did not respond with IgG2a antibody and hardly with detectable levels of IgG1. Mice immunized with a 300 fold reduced human dose (0.15 μg) alone or formulated with the Matrix M required two immunizations to develop detectable antibody responses. After two immunizations however, the Matrix M formulated D-FLU vaccine induced high antibody levels, almost as high levels (93 and 91% for IgG1 and IgG2a respectively), as the ten-fold higher dose, while the commercial vaccine alone only stimulated lower levels of IgG1 antibody, 73 and 65% compared to the Matrix adjuvanted groups and no IgG2a antibodies. No side effects were recorded in the immunized mice.

### Discussion and Conclusion

The conventional vaccine induced low levels of antibody, even with ten-fold higher doses than the Matrix M adjuvanted D-FLU. Above all, the commercial vaccine induced low quality immune responses. The low level is particularly obvious at the first immunization when the commercial vaccine formulation did not induce detectable antibody responses. An early response with good quality immune response in individuals, that are not earlier vaccinated or infected with influenza virus is important, since particularly young children being in that situation are at risk group to develop very serious illness to a natural infection with influenza virus. The low dose required of the Matrix M adjuvanted experimental vaccine to develop high levels and high quality antibodies is important not only for the fast response but above all for antigen sparing, decreasing production costs and increasing production capacity. The low quality of the response to the commercial vaccine is indicated by the lack of IgG2a. Thus, the Matrix M formulated D-FLU induced an unforeseen increase of quality to the commercial vaccine. The insufficient quality of the present FLU vaccines is well documented with regard to protection, duration of immune response requiring yearly revaccinations and quality of immune response. Besides young children, the shortcomings of poor immunogenicity of present commercially available influenza vaccines are particularly prominent in elderly. The experiments demonstrate that a Matrix M adjuvanted vaccine would fill unmet needs with regards to: (I) quality, level and duration of antibody response as well as an early effect in immunologically naìve individuals.

### EXAMPLE 3

### Disintegrated Experimental RSV Vaccine Adjuvanted with Matrix M Induces Potent Immune Protection in Cotton Rat

Respiratory syncytial virus (RSV) causes disease in man and particularly in infants and elderly, the disease can be serious. So far no therapy or vaccine is available.

In this experiment it is shown that a disintegrated RSV (DiRSV) vaccine adjuvanted with Matrix M induces a high quality immune response in cotton rats including virus neutralizing antibodies and immune protection measured as an impressive reduction in virus replication in lungs and upper respiratory tract. Cotton rats were chosen for challenge studies being an accepted model for human infection predicting the potential of a vaccine for human use.

### Experimental Layout

Cotton rats were immunized twice at days 0 and 21 with 1 μg or 5 μg doses DiRSV adjuvanted with Matrix M (24 μg/dose) in a total volume of 200 μL. The vaccine or controls (placebo or infectious virus) were injected intramuscularly (i.m.) in volumes of 100 μl in each leg of the rat (see Table 5). Animals in all groups except in group 5 were challenge infected on day 46 under light anaesthetize (Isoflurane) with a dose if 10⁵ PFU RSV strain Tracy in 100 μL.

TABLE 5
Immunization and challenge protocol of cotton rats with disintegrated
RSV (DiRSV) vaccine adjuvanted with Matrix M or controls

| | No | | |
|---|---|---|---|
| Group | rats | Treatment | Challenge on day 46 |
| 1 | 6 | Non-vaccinated animals | 10⁵ PFU RSV Tracy intranasal |
| | | injected with PBS i.m. on | |
| | | day 0 and day 21 | |
| 2 | 6 | 5 μg DiRSV antigen + | 10⁵ PFU RSV Tracy intranasal |
| | | 24 μg Matrix M i.m. on | |
| | | day 0 and day 21 | |
| 3 | 6 | 1 μg DiRSV antigen + | 10⁵ PFU RSV Tracy intranasal |
| | | 24 μg Matrix M i.m. on | |
| | | day 0 and day 21 | |
| 4 | 6 | Inoculation with | 10⁵ PFU RSV Tracy intranasal |
| | | RSV-Tracy on day 0 | |
| 5 | 6 | Naïve-no vaccine or | Not challenged |
| | | placebo | |

Bleedings for serum taken at days 0, 21, 46, and 50 were analyzed for Anti-RSV neutralizing antibodies. The rats were bled and sacrificed for virus isolation in the upper respiratory tract (URT) and the lungs at day 50.

### Results

No local or systemic side effects were recorded in any of the vaccinated animals.

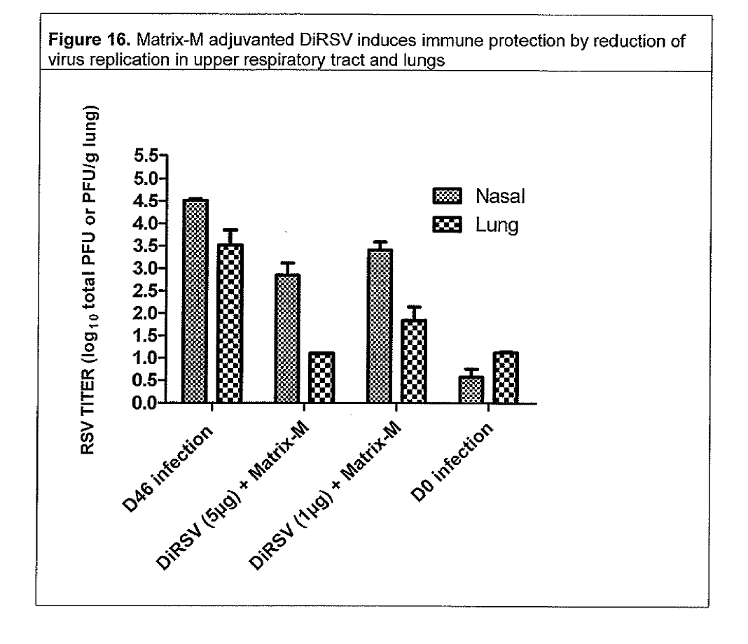

Serological responses: Virus neutralizing (VN) antibodies was measured in serum samples from all animals in each group. After one immunization, the infected animals in Group 4 responded with the highest levels of VN antibodies in serum, a level that hardly changed over time not even after challenge infection day 49. The animals immunized with the 1 and 5 μg doses of DiRSV adjuvanted with Matrix M responded with VN titers at day 21, the higher dose induced higher antibody levels. After the second immunization at day 21, the levels of VN antibodies increased and remained at this level during and after challenge infection at day 45. No anamnestic response was detected in these Groups after challenge infection (FIG. 15). Immune protection was measured by virus isolation in the upper respiratory tract and lungs, expressed as PFU in nasal wash and lung lavage (FIG. 16). The higher dose of the DiRSV Matrix M formulation induced a protective immune response in the lung that was of the same magnitude as that induced by infection i.e. 260-fold reduction of virus excretion following challenge infection compared to virus excretion in non-immunized animals. The reduction of virus replication in the URT was about 50-fold (compared to non-immunized animals). The severe symptoms following RSV infection of man is due to the viral replication in the lungs.

### Discussion and Conclusions

RSV infection causes respiratory tract infections at all ages but in infants and elderly the infections often become severe and cause morbidity and occasional mortality. There is no therapy directly targeting the virus infection. The prevention has to rely on vaccination as we understand today. However today, there is no vaccine for man against RSV infection. In the veterinary field there are vaccines but their efficacy is low and not very much used. The development of an efficient RSV vaccine is cumbersome and more than 50 years of research has not yet been accomplished with a protective vaccine. It is believed that a balanced Th1/Th2 response with IFN-y producing T cells is an important feature for the virus clearance. Earlier studies with “classical” RSV-ISCOM (incorporated RSV antigen) indicated that only mucosal (in this case intranasal) and not parental immunization was able to induce high levels of mucosal RSV specific antibody response (IgA response) in the lungs and URT (Hu et al Clin Exp Immunol 1998, Chen et al J of Irnmunol 2002, For review see, Hu et al; Adv Drug Deliv Rev 2001). The mucosal immune response is expected to exert superior protection against RSV infection. In this example, the potential of Matrix M as an adjuvant in RSV vaccines is demonstrated. Intramuscular immunization of Matrix M adjuvated DiRSV vaccine induces high levels of virus neutralization antibodies measured in serum and most importantly, high reduction of virus replication in lungs and respiratory tract after challenge infection. The cotton rat is an accepted model for human RSV infection. Thus, the observed protection in the lungs is impressive particularly regarding to the fact that virus replication in the lung is causing severe illness. Since only 5% of the DiRSV antigen constitute the protective F-protein, it is likely that other viral antigens revealed by disintegration contribute to the immune protection. Matrix M adjuvanted DiRSV vaccine is likely to fill the unmet need of a potent RSV vaccine.

### EXAMPLE 4

### Ex vivo Human Model for Evaluation of Matrix Adjuvant

In the development of vaccines for human use it is not accepted to test vaccines or their components on the natural host i.e. man. Therefore, animal models are used, which mostly are not natural host models (one exception is rabies virus vaccines) and these models have therefore limitations. Here, a new concept is introduced to get information for the development of human vaccines and in particular knowledge about the effect of adjuvants by the use of ex vivo models to study the initiation of immune responses. In that way valuable information is obtained that shorten (shortcut) the way to finalize the formulations for human use or any other species requiring such kind of information.

In this example the effect of Matrix M is analyzed on human immature dendritic cells (iDCS) being the main actors in the initiation of the immune response in man. Immature Dendritic cells (iDCs) take up and process antigens subsequently they migrate to the draining lymph node where the now matured DCs present processed antigens to lymphocytes that initiates the specific immune response. In this example we have tested the capacity of Matrix M to initiate iDCs activation measured as CD83 expression and the differentiation measured by the expression of the communication molecule CD 86 i.e. surface molecules that communicate with the lymphocytes to enter the specific immune response.

Human iDCs were obtained as described in M & M.

### Results

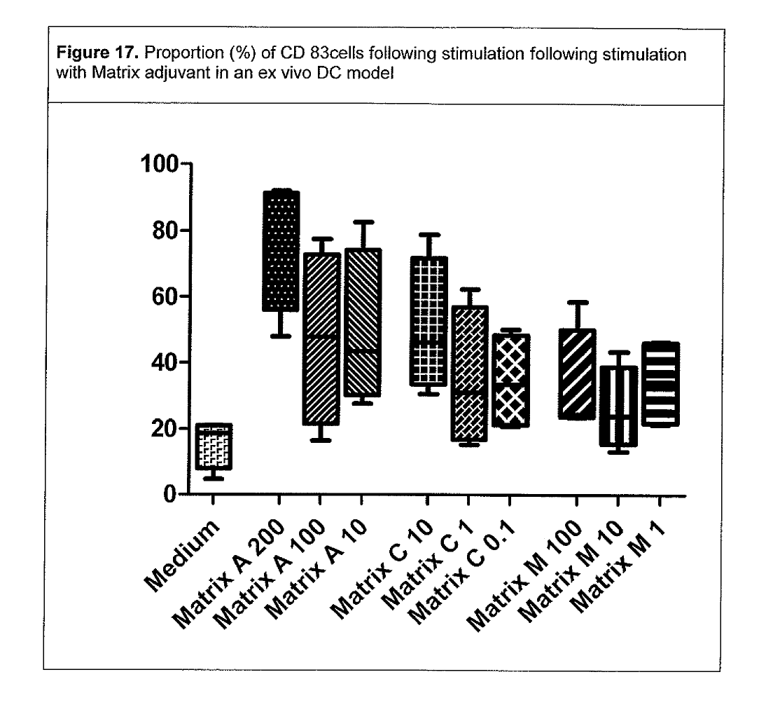

The capacity of Matrix M and its constituents Matrix A and Matrix C to activate human iDCs was evaluated by measuring the expression of CD83 after culturing iDCs with Matrix M, Matrix A and Matrix C respectively. FIG. 17 shows that both Matrix A and Matrix C induce expression of CD83 i.e. activates iDCs.

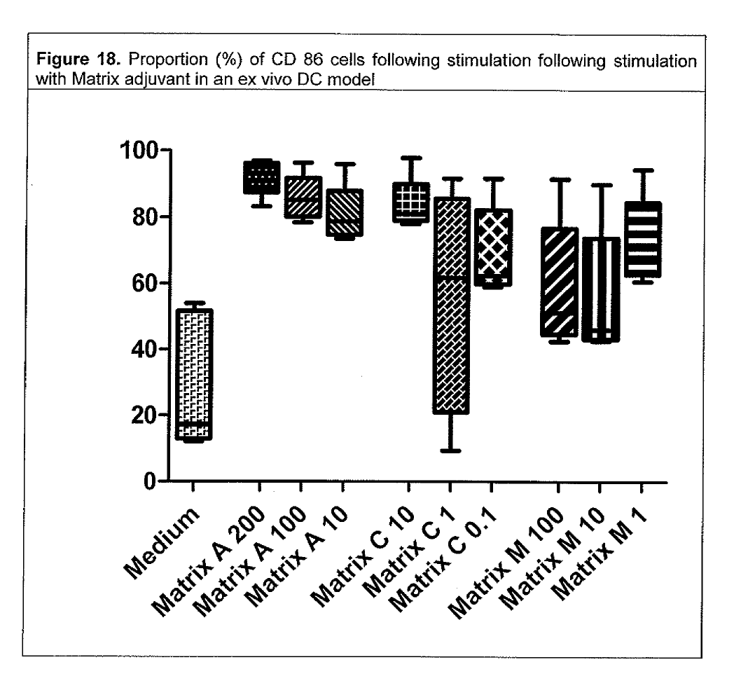

The capacity of Matrix M and its constituents Matrix A and Matrix C to differentiate human iDCs to express communication molecules was evaluated by measuring the production of CD86 after culturing iDCs with Matrix M. FIG. 18 shows that Matrix M and its constituents Matrix A and Matrix C induce expression of CD86 i.e. differentiate iDCs facilitating communication with lymphocytes.

### Discussion and Conclusion

This example clearly demonstrates the Matrix M and its constituents activates, differentiate human DCs measured by the surface molecules CD 83 and 86, which are essential for induction of acquired i.e. specific immune responses. CD 86 was selected as a read-out for this example since DCs of elderly humans are hampered in expressing this molecule resulting in immune compromised individuals prone to develop severe disease following exposure and infection with e.g. RSV or influenza virus. To conclude, we have here demonstrated that Matrix M is a novel way, facilitating vaccine development for elderly and optionally also for other immune deficiencies in man or animals.

### EXAMPLE 5

### Matrix Formulations Activates and Differentiate Dendritic Cells (DCs) Derived From Human Blood Monocytes from Elderly

RSV infects all human age groups, but causes mainly diseases in infants and elderly. Today there is no RSV vaccine available for man after more than 50 years of intensive research. After a disastrous vaccine trial in infants more than 50 years ago the research has not brought a vaccine to the market. One hurdle is that the antigen presentation, mainly exerted by DCs, is severely hampered in the elderly age group. This example demonstrates the DC hurdle of elderly is over come by ISCOM and Matrix technologies.

White blood cells were collected from ten anonymous volunteers 60 years of age or older. Monocytes were obtained and cultures in the presence of GMC-SF and IL-4 to receive immature DC (iDC) as described in Materials and Methods. The iDCs were stimulated either with; medium, LPS, Matrix M, DiRSV adjuvanted with Matrix M at two doses or an ISCOM formulation with integrated envelope proteins of RSV (described in Hu et al., Clin Exp Immunol., 113, p 325, 1998). Thereafter, the DCs were analyzed for the following surface proteins: CD11c, CD14, CD80, CD83, CD86 and (Class II antigen (major human histocompatibility complex, MHC class II) using monoclonal antibodies for identification and for quantification by flow cytometry.

### Results

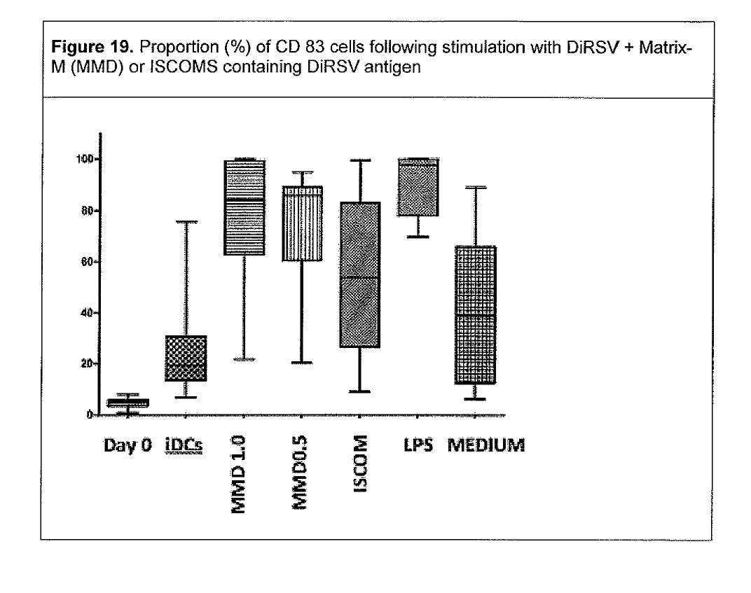

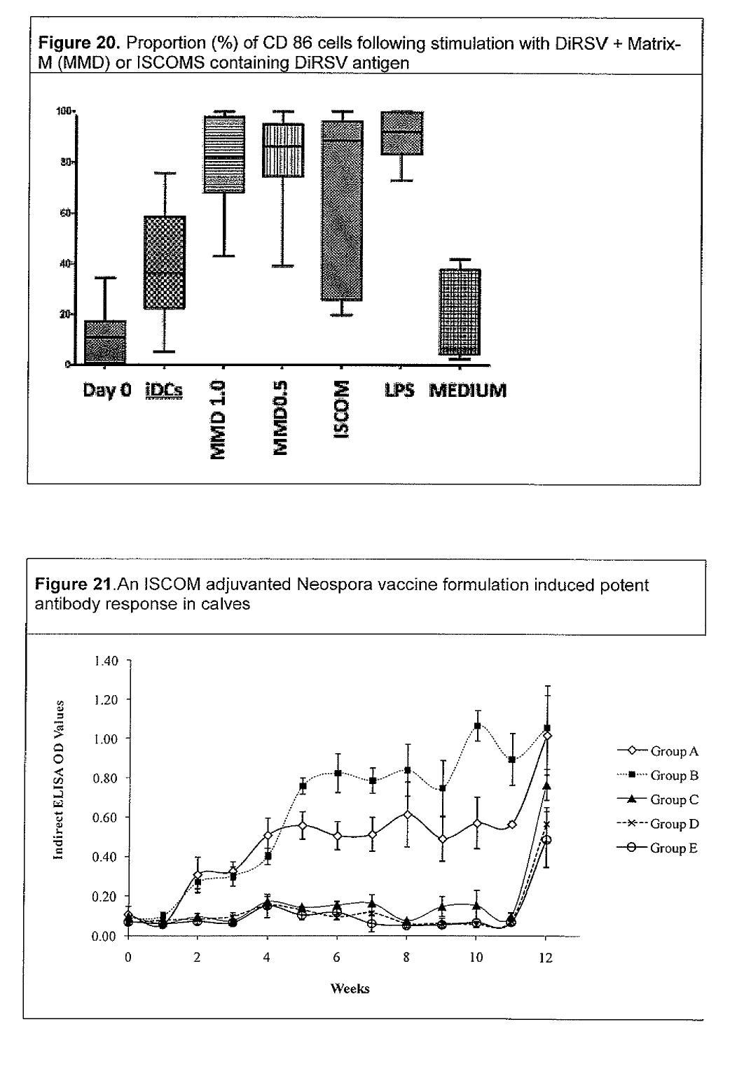

The expression of CD14 was down-regulated following culture of the monocytes, thus indicating that iDC was received. The iDCs derived from elderly volunteers were cultured for 24 h with DiRSV Matrix M vaccine or controls and then analyzed for surface receptors. After 24 h stimulation with DiRSV Matrix M vaccine the expression of activation markers on the DC were increased to the same extent as the positive control (LPS stimulation) demonstrating that the experimental vaccine activated and differentiated the iDC into activated DC. FIGS. 19 and 20 show that stimulation of iDCs with ISCOM or Matrix M adjuvanted DIRSV increased the expression of the activation marker CD83, co-stimulatory molecules CD86 and CD80 and HLADR expression. This differentiation is essential for the function of DCs to initiate the immune response to the RSV antigens.

### Discussion and Conclusion

This example shows the potential of a Matrix M based DiRSV vaccine to function also in elderly. A main reason for morbidity in elderly following RSV infection is that the elderly are immune comprised with reduced capacity of DCs to express the co-stimulatory molecules CD80 and 86 exerting communication between DCs as antigen presenting cells with lymphocytes necessary for mounting specific immune responses. Thus, newborns with an immature immune system and elderly being immune compromised by age are high risk groups for severe RSV infection due to poor capacity to develop cell mediated immunity particularly Th1responses essential for immune protection against RSV infection. Here we show in a human ex vivo model that iDC derived from monocytes from elderly, 60 years of age or older, are activated (CD83) and differentiated into mature DCs expressing CD 80 and 86 upon stimulation with an experimental vaccine DiRSV adjuvanted Matrix M or “classical” ISCOM formulations. The activation is marked by the expression of CD83 and differentiation by up-regulation of co-stimulatory molecules CD80 and CD86. Thus, these results show that “classical” ISCOM with integrated viral antigens and Matrix M adjuvanted to DiRSV have the potential to fulfill the unmet need of an effective RSV vaccine in the elderly population by overcoming the hurdle of a compromised immune system.

### EXAMPLE 6

### Disintegrated Neospora Antigens Formulated with Matrix Q Adjuvant is a Potential Vaccine Candidate

Neospora caninum (Nc) infection causes abortion and economic losses in cattle worldwide. Although there is no treatment or proven vaccine to prevent infections or disease in cattle (Dubey et al., 2007), it has been shown that cattle experimentally inoculated with live tachyzoites prior to mating developed protective immunity against vertical transmission (Innes et al., 2001). Moreover, cows with latent Neospora-infection develop protective immunity against foetopathy caused by experimental inoculation (Williams et al., 2003) or a natural second exposure to the parasite (McAllister et al., 2000). Protective mechanisms are associated with induction of type 1 immune response including IFN-γ production (Innes et al., 2002). Thus, the development of an effective vaccine should be based on an immune response that includes IFN-γ production. This example shows that the concept of using disintegrated Nc as vaccine antigen together with Matrix Q adjuvant induce high quality immune response encompassing both antibody and cell mediated immunity, particularly IFN-γ production that is expected to contribute to immune protection.

We compared some immune parameters induced in calves inoculated with live tachyzoites (proposed as a vaccine candidate) and calves inoculated with disintegrated Nc antigens adjuvanted with Matrix Q. It is shown that the experimental Matrix Q vaccine formulation gives superior immune response to the live vaccine.

### Results

Serum Antibody Responses after Immunization with Whole Live or Disintegrated Tachyzoites

Calves were inoculated i.v. once with live tachyzoits or twice s.c. with disintegrated tachyzoites either non-adjuvanted or adjuvanted with Matrix-Q as described in Table 6. Four weeks after the immunization, calves immunized with the live tachyzoits and the calves in the two Matrix Q groups developed similar levels of IgG response measured by ELISA. After the second immunization with the Matrix-Q adjuvanted disintegrated tachyzoites the calves developed significantly higher antibody responses than the calves immunized with the live tachyzoites (FIG. 21). The non-adjuvanted disintegrated tachyzoites did not induce detectable antibody responses neither after one nor two immunizations.

TABLE 6
Experimental layout

| | No | | | |
|---|---|---|---|---|
| Group | calves | Treatment | Immunization | Challenge* |
| A | 5 | Live tachyzoites (NC-1 | day 0 | week 11 |
| | | strain) 1 × 10⁸ in PBS i.v. | | |
| B | 5 | Disintegrated Neospora | day 0, | week 11 |
| | | antigens (500 μg) | week 4 | |
| | | formulated with Matrix-Q | | |
| | | (750 μg) s.c. | | |
| C | 5 | Disintegrated Neospora | day 0, | week 11 |
| | | antigens (500 μg) in PBS | week 4 | |
| | | without adjuvant s.c. | | |
| D | 5 | Matrix-Q (750 μg) in PBS | day 0, | week 11 |
| | | with no antigen s.c. | week 4 | |
| E | 5 | PBS no antigen no | day 0, | week 11 |
| | | adjuvant s.c. | week 4 | |

*All calves were challenged with 1 × 10⁸ tachyzoites of NC-1 strain by intravenous inoculation. After challenge infection at week 11 all calves developed antibodies to Neospora except those in group B. These animals in group B immunized twice with Matrix Q adjuvanted disintegrated tachyzoits did not respond to the challenge infection with increased antibody levels i.e. no anamnestic response was recorded. In contrast, animals in all other groups including the group that was immunized infection with live parasites responded to the challenge infection with increased antibody levels, i.e. an anamnestic response was recorded. An anamnestic response indicates tachyzoit replication prominent resulting increased antibody response.

IgG1 and IgG2 Antibody Responses after Various Inoculations

N. caninum specific IgG₁ and IgG₂ antibody responses presented as IgG1/IgG2 ratio are shown in Table 7. Clear differences in the distribution of the antibody response into subclasses between Groups A and B were recorded. Calves in Group A receiving live tachyzoits responded with a dominant IgG2 profile, i.e. with a ratio <1. In contrast, a ratio >1 was recorded in calves from Group B receiving disintegrated tachyzoit adjuvanted with Matrix Q. After the challenge infection (week 12) similar IgG1/IgG2 ratios were maintained in both groups.

TABLE 7
IgG1/IgG2 subclass antibodies in calves following immunization with live (A)
or Matrix-Q adjuvanted disintegrated (B) Neospora tachyzooit formulations.

| | Animal | 0 | 2 | 4 | 6 | 8 | 10 | 12 |
|---|---|---|---|---|---|---|---|---|
| Group A | 1 | 1.42 | 0.93 | 0.61 | 0.42 | 0.41 | 0.46 | 0.71 |
| | 2 | 1.71 | 0.96 | 0.78 | 0.47 | 0.34 | 0.34 | 0.83 |
| | 3 | 1.01 | 0.60 | 0.40 | 0.34 | 0.36 | 0.26 | 0.49 |
| Group B | 4 | 1.44 | 2.52 | 1.74 | 5.50 | 4.45 | 3.05 | 1.70 |
| | 5 | 1.75 | 4.15 | 2.51 | 4.53 | 2.74 | 2.78 | 1.27 |
| | 6 | 2.02 | 2.95 | 1.35 | 2.37 | 1.58 | 1.39 | 1.79 |

### IFN-γ Production in Response to Various Inoculums

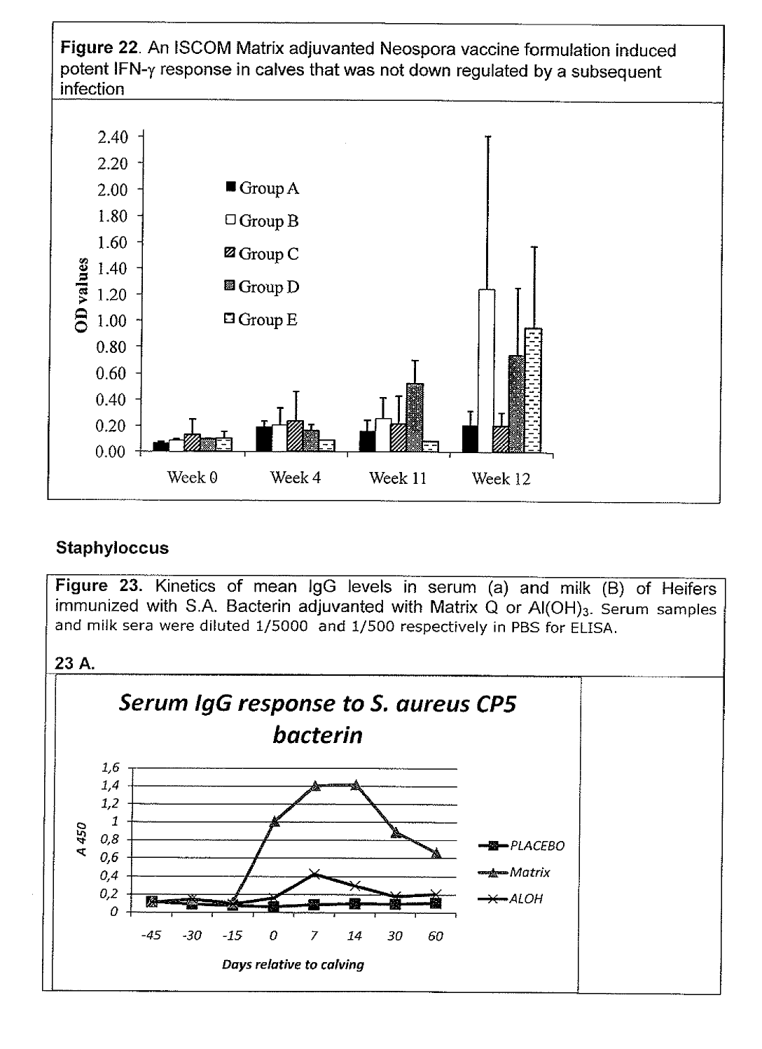

Whole blood samples from calves inoculated with various formulations of tachyzoites as described in table 6 and FIG. 22 were analyzed for the production of IFN-γ.

Specific IFN-γ responses were detected in experimentally infected cattle (Group A) and significant increases were found throughout the experiment when comparing with the level observed at week 0 (P<0.05). Maximum IFN-γ production in Group A occurred at week 4; however, it was not statistically different to the levels detected during the weeks 11 and 12 (FIG. 22) i.e. before and one week after challenge. Calves receiving disintegrated tachyzoites adjuvanted with Matrix-Q responded with increased production of IFN-γ, which was higher weeks 4 and 11 compared with week 0 (P<0.05). Interestingly, animals in this group responded to the challenge infection with increased IFN-y production observed at week 12 (P<0.05). in contrast animals immunized with non-adjuvanted live or disintegrated tachyzoites did not respond with IFN-γ production considered essential for immune protection. These results indicate that the Nc parasite antigens administered without a potent adjuvant like Matrix Q suppresses the IFN-γ response to a subsequent infection. This is supported by the notion the animals in Group D and E responded with IFN-γ productions in response to challenge infection week 11, measured week 12 (P<0.05).

### Discussions and Conclusions

In cattle, the inoculation of live tachyzoites before mating prevents not only the abortion, but also vertical transmission (Innes et al., 2001; Williams et al., 2007). However, inoculation of live to tachyzoites is not a choice for a commercial vaccine. Spread of new infections and reversion to pahogenicity are only two of many disadvantage of using live vaccines. The development of inactivated vaccines to control bovine neosporosis is still needed, commercial vaccines available today are based on inactivated whole tachyzoites and has only efficacy around 50%. The availability of inactivated immunogens that generates protective immune responses equivalent to the immunity induced by live tachyzoites is of major significance. Here we show that a vaccine based on disintegrated Neospora caninum adjuvanted with Matrix Q, induced an immune response similar to or higher (after booster) than those induced by inoculation with live tachyzoites. Calves in the group immunized with non-adjuvanted disintegrated antigens failed to induce Nc specific IgG response showing the need for a potent adjuvant. Importantly, a significant IFN-γ response, which is associated with immune protection, was observed in the group immunized with disintegrated antigens adjuvanted with Matrix-Q, which also was noted in animals inoculated with live tachyzoites. Interesting to note is that after challenge (week 12) the IFN-γ response increase to a maximum level in all groups except in the two groups receiving antigen without adjuvant. Thus, live tachyzoites and non-adjuvanted disintegrated antigens, apparently, skew or down-regulate the IFN-γ response facilitating the infection, which is overcome by Matrix adjuvant. The importance of the IFN-γ response is the notion that animals immunized with non-adjuvanted tacyzoite formulation after challenge infection developed an anamnestic response implicating infection by the parasite.

In conclusion, this study shows the potential of using disintegrated antigen of Neospora caninum together with a Matrix adjuvant in a safe and inactivated vaccine, to induce a broad immune response equivalent to the immunity induced by inoculation of live tachyzoites. However, the Matrix adjuvanted vaccine is inducing a better quality vaccine promoting IFN-γ response in contrast to a “live vaccine” formulation that down regulates the IFN-γ response occurring at a subsequent infection.

### EXAMPLE 7a

### Immune Responses to Vaccination Against a Staphylococcus aureus CP5 Bacterin in Heifers

The objective of this example was to compare the humoral immune response in serum and milk to a staphylococcal capsular polysaccharide type 5 bacterin formulated with two different adjuvants; Matrix Q or Al(OH)₃. A placebo consisting of sterile saline solution was used as control.

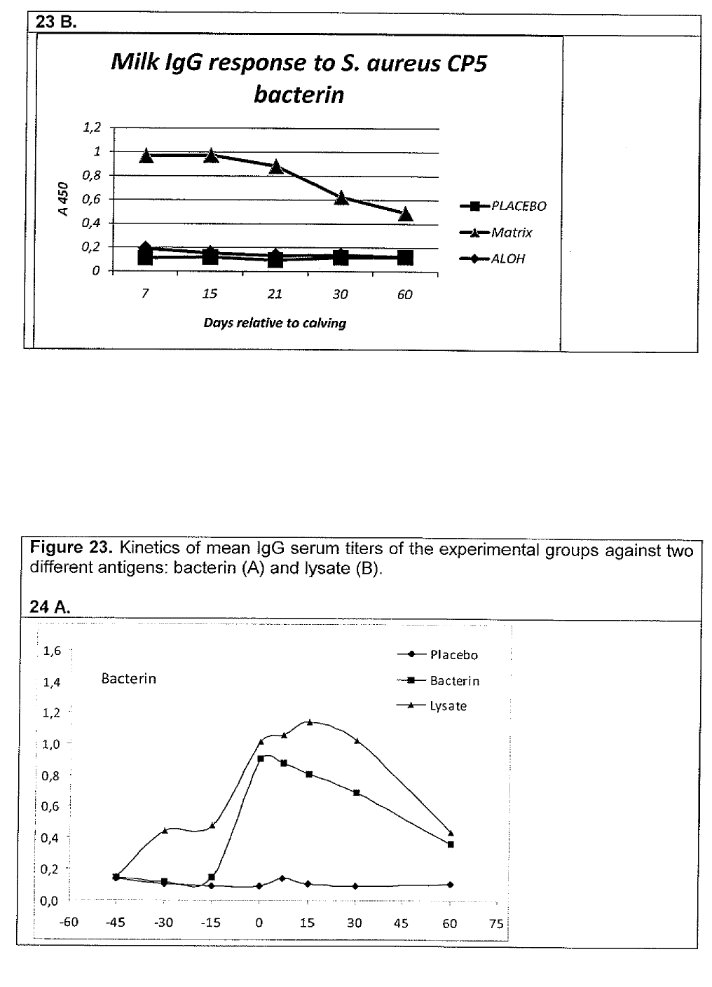

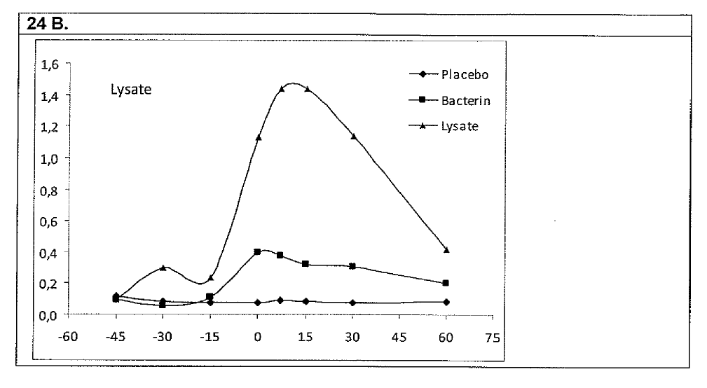

Twenty four prim gravid Holstein dairy heifers in the last trimester of gestation were used in the experiment. The animals were randomly allocated in 3 groups. Each group received one of the different formulations injected subcutaneously in the supra mammary lymph node area at approximately 45 and 15 days before expected calving. Serum and milk samples were taken as described in Table 8. The samples were tested for antibody response against whole bacteria in ELISA. The results are shown in FIGS. 23A and B.

TABLE 8
Type of sample and sampling frequency

| | Days relative | | |
|---|---|---|---|
| Number | to calving | Type of sample | Comment |
| 1 | −45 | Serum | Before 1^{st} dose |
| 2 | −30 | Serum | |
| 3 | −14 | Serum/mammary secretion | Before 2^{nd} dose |
| 4 | 0 | Serum/mammary secretion | After calving |
| 5 | +7 | Serum/milk | |
| 6 | +14 | Serum/milk | |
| 7 | +21 | Milk | |
| 8 | +30 | Serum/milk | |
| 9 | +60 | Serum/milk | |

Results and Conclusion The Matrix Q adjuvant increased the antibody response to S. Aureus CP5 bacterin in both serum and milk. The response in serum was substantially higher and of longer duration. In milk, Al(OH)₃ did not at al promote an antibody response. In contrast, Matrix Q adjuvanted Bacterin stimulated to high levels of antibodies in milk. The bacterin without any adjuvant did not induce any detectable antibodies at all.

### EXAMPLE 7b

### Humoral Immune Responses to Vaccination Against Staphylococcus aureus CP5 Bacterin and CP5 Lysate in Heifers

The objective of this example was to evaluate the humoral response generated by (1) a Staphylococcus aureus CP5 whole cell vaccine and (2) a S. aureus CP5 lysate vaccine, both formulated with Matrix Q adjuvant and (3) a placebo consisting of sterile saline solution plus Matrix-Q adjuvant was used as control. The vaccines are formulated as described in M&M. Twelve primigravid Holstein dairy heifers in the last trimester of gestation were used in the experiment. The animals were randomly allocated in 3 groups. Each group received one of the different formulations injected subcutaneously in the supramammary lymph node area at approximately 40 and 14 days before expected calving. Samples were taken as described in Table 9. The sera were tested for antibody response against whole bacteria and a bacteria lysate (disintegrated bacteria) in ELISA. The results are shown in FIGS. 24A and B.

TABLE 9
Type of sample and sampling frequency

| | Days relative to | | |
|---|---|---|---|
| Number | calving | Type of sample | Comment |
| 1 | −45 | Serum | Before 1^{st} dose |
| 2 | −30 | Serum | Before 2^{nd} dose |
| 3 | −15 | | |
| 4 | 0 | Serum | After calving |
| 5 | +7 | Serum | |
| 6 | +15 | Serum | |
| 7 | +30 | Serum | |
| 8 | +60 | Serum | |

### Results and Conclusion

The results of the study are shown in FIG. 23. The serum antibody responses are higher in heifers that received the disintegrated SA experimental vaccine, regardless if measured against SA. antigen in Bacterin or Lysate form. Mean sera IgG titers of the three experimental groups are shown in FIG. 23. When the antibody response is measured against SA lysate, exposing also internal antigens, the response is substantially higher if bacterial lysate was used for immunization, demonstrating that a whole range of additional antigens have become antigenic. In conclusion it is demonstrated that Matrix M in combination with disintegrated S.A. cells enhances and broaden the antibody response compared to Matrix M adjuvanted non-disintegrated cells not to mention non-adjuvanted SA whole cell and that the potent and immune modulating capacity of Matrix M is essential.

1. Composition comprising at least one ISCOM complex and at least one internal antigen which is not a surface antigen and not in the form of a part of a whole micro-organism.

2. The composition according to claim 1, wherein the at least one internal antigen is in the form of a whole micro-organism.

3. The composition according to claim 1, wherein the at least one internal antigen is a nucleoprotein.

4. The composition according to claims 1 and 3, wherein the least one internal antigen is a member of the group of components obtained after disintegrating a micro-organism.

5. The composition according to claim 3, wherein the least one internal antigen is a member of the group of components obtained after disintegrating a micro-organism with a solubilising agent.

6. The composition according to claim 5, wherein the solubilising agent in the composition has been diluted 2-100 times after the disintegration.

7. The composition according to any of claims 1-6, wherein the ISCOM complex is an ISCOM comprising at least one saponin, at least one lipid and at least one antigen.

8. The composition according to any of claims 1-6, wherein the ISCOM complex is an ISCOM matrix comprising at least one saponin, and at least one lipid.

9. The composition according to claims 7 and 8, wherein the at least one lipid is cholesterol and at least one phospolipid.

10. The composition according to any of claims 1-9, wherein the sapoin is chosen from crude saponin extract of Quillaja saponaria Molina, fraction Q and Q-VAC; the C, the B, B3, B4 and B4b and QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20 and QA-22 fractions of Quillaja saponaria Molina.

11. The composition according to any of claims 1-10, wherein the composition comprises at least two different immunogenic complexes chosen from ISCOM complexes and/or ISCOM-matrix complexes, each individual complex comprising one saponin fraction from Quillaja Saponaria Molina, wherein the saponin fraction in one complex is different from the saponin fraction in the other complex.

12. The composition according to any of claim 1-11, wherein the internal antigen is chosen from internal components in micro-organisms such as virus, bacteria, parasites, yeast cells, eukaryotic cells, including mammalian cells, insect cells.

13. The composition according to claim 12, wherein the internal antigen derive from RSV, Rabies virus, influenza virus, Staphylococcus aureus and Neopsora.

14. The composition according to any of claims 1-13 at least one ISCOM complex, further also comprising non internal antigens.

15. The composition according to any of claim 1-14, further also comprising whole microorganisms which may be live and attenuated.

16. The composition according to any of claim 1-15, further also comprising one or more additives pharmaceutically acceptable excipients, carriers and/or diluents.

17. A composition according to any of claims 1-16 for use as an immune stimulating medicine or vaccine.

18. The composition according to claim 17 for use in eliciting T cell respond including CTL respond.

19. The composition according to any of claims 17 and 18 for use as an immune stimulating medicine or vaccine for low responders.

20. The composition according to any of claims 17 -19, wherein the low responders are, sick people, elderly people or juveniles.

21. A process for preparing a composition comprising at least one ISCOM complex and at least one internal antigen, which is not a surface antigen and not in the form of a part of a whole imicro-organism, characterized in that a saponin, cholesterol and a lipid are mixed with a lysed cell suspension of cells and solubilising agent without removal of any cell components, whereafter the solubilising agent is removed or diluted.

22. Kit comprising at least two compartments, wherein one compartment comprises an ISCOM complex comprising at least one internal antigen, which is not a surface antigen and not in the form of a part of a whole micro-organism and the other compartment comprises a prescription for use or wherein
the first compartment comprises an ISCOM matrix complex and the other compartment comprises at least one internal antigen, which is not a surface antigen and not in the form of a part of a whole micro-organism.

23. The kit according to claim 22, wherein the at least one internal antigen is a member of the group of components obtained after disintegrating a micro-organism.

24. A composition comprising at least one ISCOM complex for use as an immune stimulating or immune modulating medicine or vaccine for the stimulation of dendritic cells in immunologically low responders such as juveniles, infants or elderly.

25. The composition according to claim 24 wherein the dendritic cells are chosen from CD 80, CD 83, CD 86 and chimocine CCR 227.

26. The composition according to any of claims 24 and 25 wherein the immunologically low responder is a mammal especially a human.

27. The composition according to any of claims 24 and 25, wherein the ISCOM complex is according to any of claims 7-11.

28. The composition according to any of claims 24-27, further also comprising one or more additives accords to claim 16.
